(19) United States
(12) Patent Application Publication
(10) Pub. No.: US 2023/0139593 A1
(43) Pub. Date: May 4, 2023

(54) TREATMENT OF DISORDERS ASSOCIATED WITH OXIDATIVE STRESS AND COMPOUNDS FOR SAME

(71) Applicants: The University of Adelaide, North Terrace (AU); The Board of Regents of the University of Texas System, Austin, TX (US)

(72) Inventors: Thomas Avery, North Terrace (AU); Andrew Abell, North Terrace (AU); Dion Turner, North Terrace (AU); Peter M. Grace, Houston, TX (US); Jiahe Li, Houston, TX (US)

(21) Appl. No.: 17/911,346

(22) PCT Filed: Mar. 12, 2021
(86) PCT No.: PCT/AU2021/050217

§ 371 Date: Sep. 13, 2022

Related U.S. Application Data
(60) Provisional application No. 62/989,324, filed on Mar. 13, 2020.

(51) Int. Cl. A61K 31/216; A61K 31/22; A61K 31/40; A61K 31/4409; A61K 31/341; A61P 25/02

(52) U.S. Cl. CPC A61K 31/216; A61K 31/22; A61K 31/40; A61K 31/4409; A61K 31/341; A61P 25/02

(57) ABSTRACT

The present invention relates to the treatment of disorders associated with oxidative stress including neuropathic pain and small synthetically derived compounds for treating such disorders.

### FIELD

The present invention relates to the treatment of disorders associated with oxidative stress including neuropathic pain and small synthetically derived compounds for treating such disorders.

### BACKGROUND

Oxidative stress occurs when there is an imbalance between formation and regulation of reactive oxygen (ROS) and nitrogen species (RNS) in cells and tissues, causing ROS and RNS to accumulate. This imbalance leads to damage of important biomolecules and cells, with potential impact on the whole organism. Oxidative stress influences many physiological processes including the immune system and cellular communication, and has been linked to intense exercise, inadequate diet, ageing and several age-related disorders, as well as many chronic diseases. Some of the chronic diseases shown to be associated with increased levels of oxidative stress include; cardiovascular diseases, including vascular diseases like atherosclerosis, high cholesterol, stroke, heart failure, and hypertension; cancer; Parkinson's disease; Alzheimer's disease; diabetes; pain disorders; multiple sclerosis; kidney disease; rheumatoid arthritis; sepsis; respiratory distress syndrome, and metabolic disorders such as mitochondrial diseases, lipid metabolism disorders, and DNA repair-deficiency disorders. Other conditions may include chronic obstructive pulmonary disease (COPD) which includes conditions emphysema, chronic bronchitis & chronic asthma and chronic kidney disease (CKD).

For instance, neuropathic pain is also a disorder linked to oxidative stress and is caused by nervous system lesion or disease. It has an estimated prevalence of 7-10% in the general population and is a tremendous burden to the economy and the patient's quality of life. Pharmacological treatment of such pain relies primarily upon monoamine reuptake inhibitors, anticonvulsant agents, and opioids. First line treatments include amitriptyline, duloxetine, gabapentin and pregabalin. Such drugs have only modest efficacy and are also plagued by adverse effects and risk for misuse and abuse. Several strategies have been proposed to realize new and non-addictive treatments for chronic pain, including development of drugs that target endogenous pain-resolution mechanisms and that simultaneously modify multiple pathophysiological mechanisms that underlie pain. There have been many attempts to pharmacologically control oxidative stress in chronic disease states (for instance pain) to date, none are in clinical use. Supplementation of individual antioxidants may have not only failed due to unfavourable pharmacokinetics, but also because numerous antioxidants are required to restore homeostasis by collaboratively catabolizing reactive oxygen species.

The present invention seeks to address some of the shortcomings of the prior art therapeutics and is directed to a specific class of compounds which target a particular regulator of the antioxidant response and are now shown, for the first time, to be useful in the treatment of disorders associated with oxidative stress, such as the alleviation of pain, for instance, neuropathic pain.

### SUMMARY

In one aspect the invention provides a method of treating disorders associate with oxidative stress including the step of administering to a subject in need thereof a compound of formula (I):

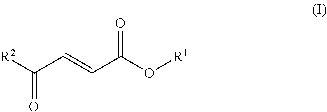

wherein
R¹ is selected from C₁-C₃ alkyl;
R² represents:

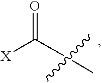

where X is selected from OH, OX¹, CH₂C(O)X², C(O)X², CHCHC(O)X², optionally substituted aryl, optionally substituted heteroaryl, optionally substituted heterocyclyl, optionally substituted alkyl, optionally substituted alkoxy, optionally substituted amino and optionally substituted thio; and
wherein X¹ is selected from optionally substituted aryl, optionally substituted heteroaryl, optionally substituted heterocyclyl, optionally substituted alkyl, optionally substituted amino and optionally substituted thio; and
wherein X² is selected from OH, optionally substituted aryl, optionally substituted heteroaryl, optionally substituted heterocyclyl, optionally substituted alkyl, optionally substituted alkoxy, optionally substituted amino and optionally substituted thio;
or a pharmaceutically acceptable salt, solvate, or isomer thereof.

In an embodiment the method relates to the treatment of vascular diseases like atherosclerosis, high cholesterol, stroke, heart failure, ischemia-reperfusion injury and hypertension; cancer; neurodegenerative diseases (such as Parkinson's disease (PD), Alzheimer's disease (AD) amyotrophic lateral sclerosis (ALS)); diabetes; pain disorders in particular those related to inflammatory pain, neuropathic pain, visceral pain, migraine, or unknown origin (such as complex regional pain syndrome, fibromyalgia); multiple sclerosis; kidney disease; rheumatoid arthritis; sepsis; respiratory distress syndrome; and metabolic disorders, such as mitochondrial diseases, lipid metabolism disorders, and DNA repair-deficiency disorders.

In an embodiment the method relates to the treatment of ischemia-reperfusion injury.

In an embodiment the method relates to the treatment of pain and, in particular, neuropathic pain.

In an embodiment the method relates to the treatment of peripheral neuropathic pain.

In an embodiment the method relates to the treatment of chronic obstructive pulmonary disease (COPD)—which includes conditions emphysema, chronic bronchitis & chronic asthma or chronic kidney disease (CKD).

In a further aspect the invention provides the use of compounds of formula (I) for treating disorders associate with oxidative stress.

In still a further aspect the invention provides the use of compounds of formula (I) in the manufacture of a medicament for treating disorders associate with oxidative stress.

In certain embodiments the uses above relate to the treatment of ischemia-reperfusion injury.

In certain embodiments the uses above relate to the treatment of pain and, in particular, neuropathic pain.

In certain embodiments the uses above relate to the treatment of peripheral neuropathic pain.

In certain embodiments the uses above relate to the treatment of chronic obstructive pulmonary disease (COPD)—which includes conditions emphysema, chronic bronchitis & chronic asthma or chronic kidney disease (CKD).

### DESCRIPTION OF THE FIGURES

FIG. 1. In vitro characterization of examples of compounds described in the invention. (a-c) NRF2/ARE luciferase reporter HEK293 cells were treated with media, H₂O₂ (10 μM), or ONOO⁻ (10 μM), followed by MMF or 1,2-dicarbonyl compounds. MMF increased luciferase activity in a concentration dependent manner (P<0.001), and activity was not influenced by H₂O₂ or ONOO⁻ (P=0.256). (a) 1,5-dimethyl (2E)-4-oxopent-2-enedioate increased NRF2 activity in the presence of H₂O₂ or ONOO⁻, compared to media control, at comparable levels to MMF. (b) Although methyl (2E)-4-(benzylcarbamoyl)-4-oxobut-2-enoate activated NRF2 to a greater extent than MMF, methyl (2E)-4-(benzylcarbamoyl)-4-oxobut-2-enoate did not selectively increase NRF2 activity when co-incubated with H₂O₂ or ONOO⁻, compared to media (P=0.288). Methyl (2E)-4-(benzylcarbamoyl)-4-oxobut-2-enoate was also cytotoxic above 50 μM. (c) Methyl (2E)-4,5-dioxo-5-phenylpent-2-enoate increased NRF2 activity in the presence of H₂O₂ or ONOO⁻, compared to media control, but at lower levels than MMF. Not significant (n.s.), **P<0.01, ***P<0.001.

FIG. 2. Behavioral assessment of examples of compounds described in the invention (a, b) Once neuropathic pain was established, male and female mice were orally treated with 1,5-dimethyl (2E)-4-oxopent-2-enedioate (350 μmol/kg), diroximel fumarate (350 μmol/kg), or vehicle every day for 3 days (gray box). (a) Tactile allodynia and (b) dynamic allodynia were assessed. (c) Once neuropathic pain was established, male and female mice were orally treated with 1,5-dimethyl (2E)-4-oxopent-2-enedioate (350 μmol/kg or vehicle every day for 7 days. Spontaneous pain was measured by the conditioned place preference test after 7 days of 1,5-dimethyl (2E)-4-oxopent-2-enedioate treatment. The Y axis indicates the difference between time spent in light chamber prior to treatment and after 7 days of treatment. Relative to vehicle: *P<0.05, **P<0.01, ****P<0.0001; 1,5-dimethyl (2E)-4-oxopent-2-enedioate vs. diroximel fumarate: ##P<0.05, ###P<0.01, ##P<0.0001. (d, e) Once neuropathic pain was established, male and female mice were orally treated with (3E)-5-methoxy-2,5-dioxopent-3-enoic acid (350 μmol/kg) or vehicle every day for 3 days (gray box), and (d) tactile allodynia and (e) dynamic allodynia were assessed. Relative to vehicle: **P<0.01, ****P<0.0001.

FIG. 3. Evaluation of in vivo site-specific cleavage of examples of compounds described in the invention (a,b). (a) Schematic of injury site and tissues of interest. (b) Once neuropathic pain was established, male and female mice were orally treated with 1,5-dimethyl (2E)-4-oxopent-2-enedioate (350 μmol/kg), diroximel fumarate (350 μmol/kg), or vehicle every day for 3 days. L4/5 DRG from 3 mice were pooled after 3 days of treatment, and nuclear extracts were probed for NRF2 (n=2 males, n=2 females). Relative to ipsilateral vehicle: ****P<0.0001; relative to contralateral vehicle and 1,5-dimethyl (2E)-4-oxopent-2-enedioate: ^{††††}P<0.0001.

FIG. 4. Absolute leukocyte count. Naive male and female mice were orally treated with 1,5-dimethyl (2E)-4-oxopent-2-enedioate (350 μmol/kg), diroximel fumarate (350 μmol/kg), or vehicle every day for 10 days. Blood was collected by cardiac puncture and leukocytes were manually counted.

### DETAILED DESCRIPTION OF THE INVENTION

Without wishing to be bound by the theory the present inventors have identified that certain compounds disclosed herein (compounds of formula (I)) are able to be effectively cleaved in vivo in the presence of endogenous peroxides (or other ROS/RNS species) for example, hydrogen peroxide (HP) and/or peroxynitrite (PN), to generate the active drug molecule monomethyl fumarate (MMF) or R¹ alkyl esters thereof.

The inventors have devised novel 1,2-dicarbonyl or 1,2,3-tricarbonyl molecular frameworks which release an activator of the NRF2 pathway, namely, monomethyl fumarate (MMF), in response to HP and PN. For example, 1,2-dicarbonyl systems (see Scheme 1 below) have been shown (HPLC analysis) by the inventors to react with high conversion to form anhydrides on treatment with HP and PN, in a Baeyer-Villiger like reaction, with no observable side by-products owing to the two potential acylium intermediates having the highest migratory aptitudes. The transient anhydride formed is highly susceptible to hydrolysis in an aqueous/physiological environment. The inventors have shown release of MMF on treatment with HP in high yield, with several chemical subclasses, in a phosphate buffer system at pH 7.4. This framework is robust, testable and provides latitude for electronic tuning of the reactivity of the system owing to its conjugated nature.

In disorders associated with oxidative stress, the present investors have recognized that peroxide oxidants such as HP and PN are elevated in certain cells, tissues and biological compartments, as such circulating compounds of formula (I) will release MMF stoichiometrically in line with the amount of oxidant and therefore activate the NRF2 pathway at the site best suited to effect amelioration of the disorder by restoring redox balance in regions specifically under oxidative stress. This tissue targeting concept has for the first time been evidenced by the inventors in efficacy models of neuropathic pain. Furthermore, systemic delivery of MMF has been recognized to cause certain problems, one of them is reduced lymphocyte count. The present inventors have obtained data showing that the compounds disclosed herein do not lead to reduced lymphocyte count.

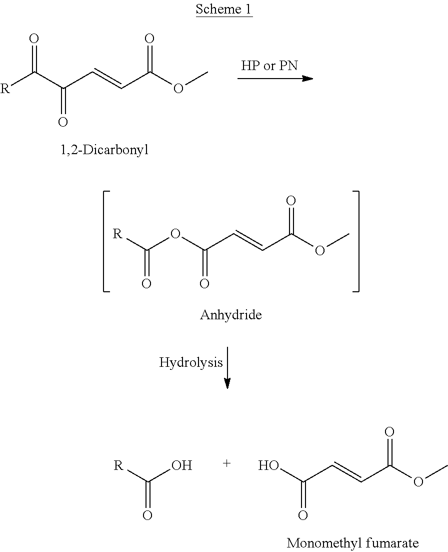

In further aspects of the invention there is provided pharmaceutical compositions for treating disorders associated with oxidative stress, the composition comprising an effective amount of a compound of formula (I) as herein defined or a pharmaceutically acceptable salt thereof and optionally a carrier or diluent.

In certain embodiments the therapeutic methods and uses disclosed herein utilize compounds of formula (I) as represented by formula (Ia):

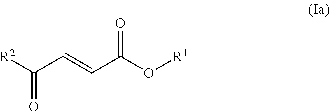

where R¹ is C₁-C₃ alkyl and
where R² is selected from:
a)

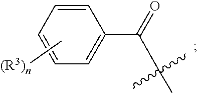

b) optionally substituted C₁-C₁₂ alkyl

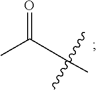 [SMILES: *C(C)=O]

c) optionally substituted heterocyclyl

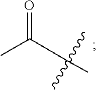 [SMILES: *C(C)=O]

d) optionally substituted heteroaryl

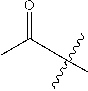 [SMILES: *C(C)=O]

e)

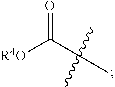

f)

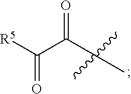

g)

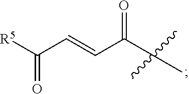

h)

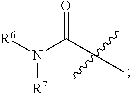

and
i)

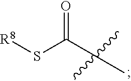

wherein R³ is selected from OH, CI, F, CF₃, CN, OCF₃, optionally substituted alkyl, optionally substituted alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, optionally substituted cycloalkyl, optionally substituted cycloalkyloxy, optionally substituted sulfinyl, and optionally substituted sulfonyl,
n is an integer selected from 0 to 3;
R⁴ is selected from H, optionally substituted alkyl, optionally substituted aryl, optionally substituted heteroaryl, and optionally substituted heterocyclyl;
R⁵ is selected from optionally substituted alkyl, optionally substituted alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, and optionally substituted cycloalkyl;
R⁶ and R⁷ are independently selected from H, optionally substituted C₁-C₆ alkyl, optionally substituted aryl and optionally substituted arylalkyl; and
R⁸ is selected from optionally substituted alkyl, optionally substituted aryl, and optionally and substituted arylalkyl.

Representative compounds contemplated for use in the treatment methods disclosed herein include:

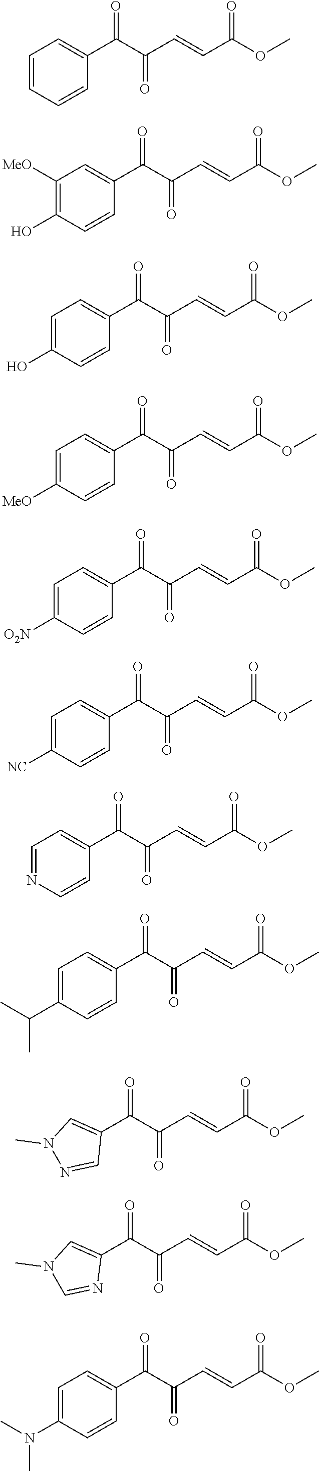

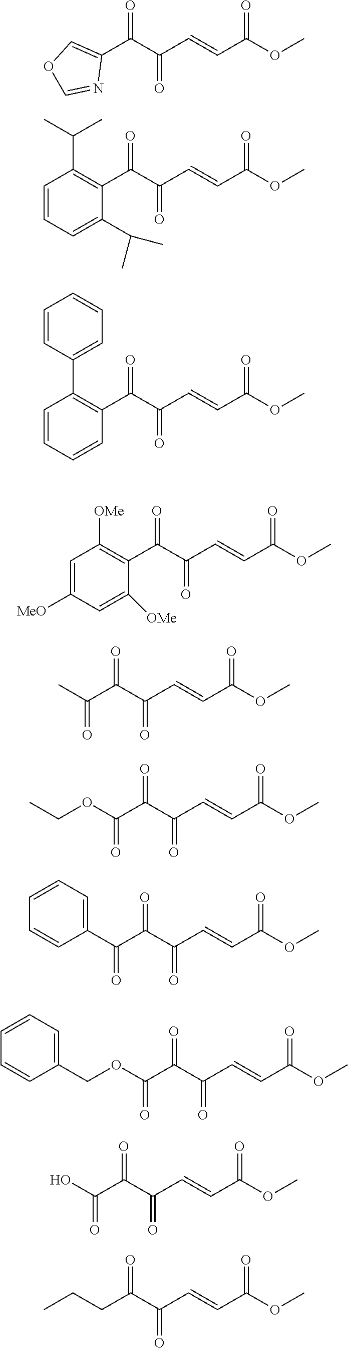

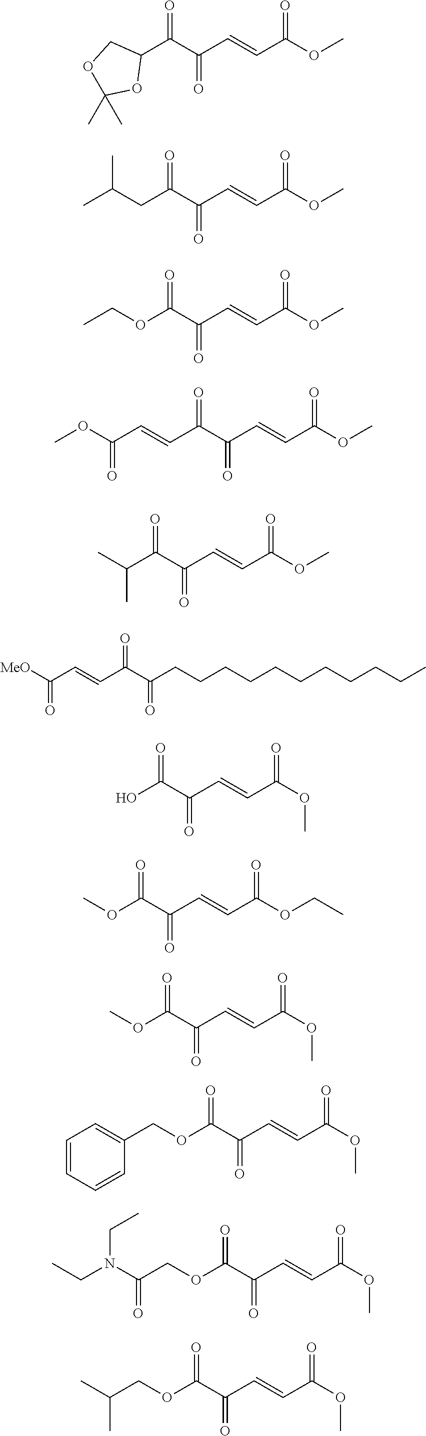

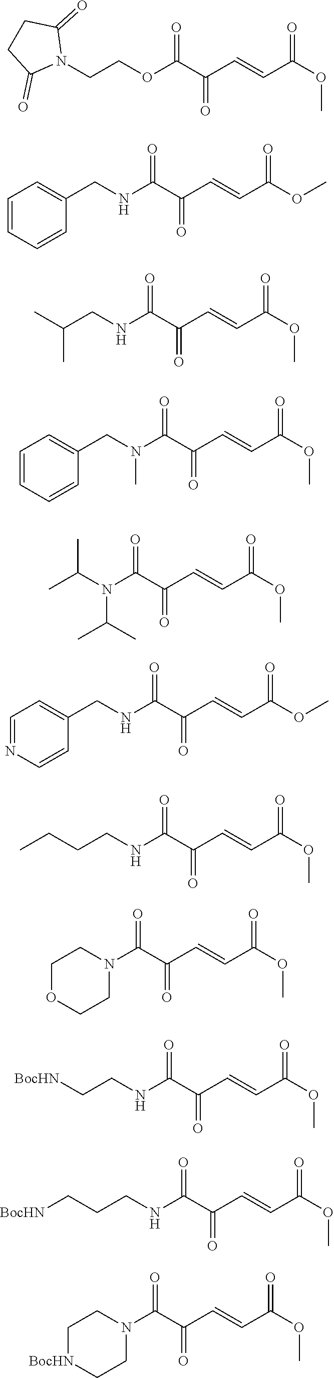

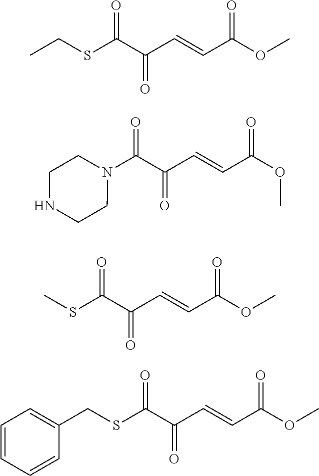

In still a further aspect the invention provides compounds of formula (II)

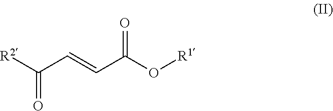

or pharmaceutically acceptable salts thereof:
where R^{1′} is C₁-C₃ alkyl and
where R^{2′} is selected from:
a

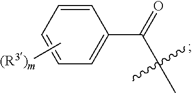

b′) optionally substituted C₂-C₁₀ alkyl

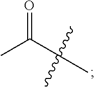 [SMILES: *C(C)=O]

c′) optionally substituted heterocyclyl

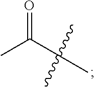 [SMILES: *C(C)=O]

d′) optionally substituted heteroaryl

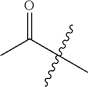 [SMILES: *C(C)=O]

e′)

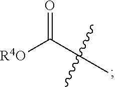

f′)

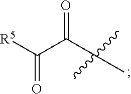

g′)

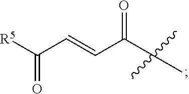

h′)

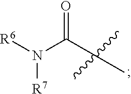

and
i′)

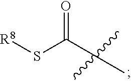

wherein R^{3′} is selected from OH, CI, F, CF₃, CN, OCF₃, optionally substituted alkyl, optionally substituted alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, optionally substituted cycloalkyl, optionally substituted cycloalkyloxy, optionally substituted sulfinyl, and optionally substituted sulfonyl;
m is an integer selected from 1 to 3;
provided that when m is 1, R^{3′} is not methyl, OH, NO₂ or methoxy;
R^{4′} is selected from optionally substituted C₄-C₈ alkyl, optionally substituted aryl, optionally substituted heteroaryl, and optionally substituted heterocyclyl;
R^{5′} is selected from optionally substituted alkyl, optionally substituted C₂-C₆ alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, and optionally substituted cycloalkyl;
R^{6′} and R^{7′} are independently selected from H, optionally substituted C₁-C₆ alkyl, optionally substituted aryl and optionally substituted arylalkyl; provided that when one of R^{6′} or R^{7′} is H the other is not benzyl; and
R^{8′} is selected from optionally substituted alkyl, optionally substituted aryl, and optionally and substituted arylalkyl.

### Definitions

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Aryl” refers to an unsaturated aromatic carbocyclic group having a single ring (e.g., phenyl) or multiple condensed rings (e.g., naphthyl or anthryl), preferably having from 6 to 14 carbon atoms. Examples of aryl groups include phenyl, naphthyl and the like.

“Aryloxy” refers to the group aryl-O— wherein the aryl group is as described above.

“Arylalkyl” refers to -alkylene-aryl groups preferably having from 1 to 10 carbon atoms in the alkylene moiety and from 6 to 10 carbon atoms in the aryl moiety. Such arylalkyl groups are exemplified by benzyl, phenethyl and the like.

“Arylalkoxy” refers to the group arylalkyl-O— wherein the arylalkyl group are as described above. Such arylalkoxy groups are exemplified by benzyloxy and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Alkenyloxy” refers to the group alkenyl-O— wherein the alkenyl group is as described above.

“Alkynyl” refers to alkynyl groups preferably having from 2 to 10 carbon atoms and more preferably 2 to 6 carbon atoms and having at least 1, and preferably from 1-2, carbon to carbon, triple bonds. Examples of alkynyl groups include ethynyl (—C≡CH), propargyl (—CH₂C≡CH), pent-2-ynyl (—CH₂C≡CCH₂—CH₃), and the like.

“Alkynyloxy” refers to the group alkynyl-O— wherein the alkynyl groups is as described above.

“Acyl” refers to groups H—C(O)—, alkyl-C(O)—, cycloalkyl-C(O)—, aryl-C(O)—, heteroaryl-C(O)— and heterocyclyl-C(O)—, where alkyl, cycloalkyl, aryl, heteroaryl and heterocyclyl are as described herein.

“Oxyacyl” refers to groups HOC(O)—, alkyl-OC(O)—, cycloalkyl-OC(O)—, aryl-OC(O)—, heteroaryl-OC(O)—, and heterocyclyl-OC(O)—, where alkyl, cycloalkyl, aryl, heteroaryl and heterocyclyl are as described herein.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminoacyl” refers to the group —C(O)NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Acylamino” refers to the group —NR″C(O)R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyloxy” refers to the groups —OC(O)-alkyl, —OC(O)-aryl, —C(O)O-heteroaryl, and —C(O)O-heterocyclyl where alkyl, aryl, heteroaryl and heterocyclyl are as described herein.

“Aminoacyloxy” refers to the groups —OC(O)NR″-alkyl, —OC(O)NR″-aryl, —OC(O)NR″-heteroaryl, and —OC(O)NR″-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacylamino” refers to the groups —NR″C(O)O-alkyl, —NR″C(O)O-aryl, —NR″C(O)O-heteroaryl, and NR″C(O)O-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacyloxy” refers to the groups —OC(O)O-alkyl, —O—C(O)O-aryl, —OC(O)O— heteroaryl, and —OC(O)O-heterocyclyl where alkyl, cycloalkyl, aryl, heteroaryl, and heterocyclyl are as described herein.

“Acylimino” refers to the groups —C(NR″)—R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyliminoxy” refers to the groups —O—C(NR″)—R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Oxyacylimino” refers to the groups —C(NR″)—OR″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, indanyl, 1,2,3,4-tetrahydronapthalenyl and the like.

“Cycloalkenyl” refers to cyclic alkenyl groups having a single cyclic ring or multiple condensed rings, and at least one point of internal unsaturation, preferably incorporating 4 to 11 carbon atoms. Examples of suitable cycloalkenyl groups include, for instance, cyclobut-2-enyl, cyclopent-3-enyl, cyclohex-4-enyl, cyclooct-3-enyl, indenyl and the like.

“Halo” or “halogen” refers to fluoro, chloro, bromo and iodo.

“Heteroaryl” refers to a monovalent aromatic heterocyclic group which fulfils the Hückel criteria for aromaticity (i.e., contains 4n+2π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (e.g., pyridyl, pyrrolyl or N-oxides thereof or furyl) or multiple condensed rings (e.g., indolizinyl, benzoimidazolyl, coumarinyl, quinolinyl, isoquinolinyl or benzothienyl). It will be understood that where, for instance, R² or R′ is an optionally substituted heteroaryl which has one or more ring heteroatoms, the heteroaryl group can be connected to the core molecule of the compounds of the present invention, through a C—C or C-heteroatom bond, in particular a C—N bond.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Thio” refers to groups H—S—, alkyl-S—, cycloalkyl-S—, aryl-S—, heteroaryl-S—, and heterocyclyl-S—, where alkyl, cycloalkyl, aryl, heteroaryl and heterocyclyl are as described herein.

“Thioacyl” refers to groups H—C(S)—, alkyl-C(S)—, cycloalkyl-C(S)—, aryl-C(S)—, heteroaryl-C(S)—, and heterocyclyl-C(S)—, where alkyl, cycloalkyl, aryl, heteroaryl and heterocyclyl are as described herein.

“Oxythioacyl” refers to groups HO—C(S)—, alkylO—C(S)—, cycloalkylO—C(S)—, arylO—C(S)—, heteroarylO—C(S)—, and heterocyclylO—C(S)—, where alkyl, cycloalkyl, aryl, heteroaryl and heterocyclyl are as described herein.

“Oxythioacyloxy” refers to groups HO—C(S)—O—, alkylO—C(S)—O—, cycloalkylO—C(S)—O—, arylO—C(S)—O—, heteroarylO—C(S)—O—, and heterocyclylO—C(S)—O—, where alkyl, cycloalkyl, aryl, heteroaryl and heterocyclyl are as described herein.

“Phosphorylamino” refers to the groups —NR″—P(O)(R′″)(OR″″) where R″ represents H, alkyl, cycloalkyl, alkenyl, or aryl, R′″ represents OR″″ or is hydroxy or amino and R″″ is alkyl, cycloalkyl, aryl or arylalkyl, where alkyl, amino, alkenyl, aryl, cycloalkyl, and arylalkyl are as described herein.

“Thioacyloxy” refers to groups H—C(S)—O—, alkyl-C(S)—O—, cycloalkyl-C(S)—O—, aryl-C(S)—O—, heteroaryl-C(S)—O—, and heterocyclyl-C(S)—O—, where alkyl, cycloalkyl, aryl, heteroaryl, and heterocyclyl are as described herein.

“Sulfinyl” refers to groups H—S(O)—, alkyl-S(O)—, cycloalkyl-S(O)—, aryl-S(O)—, heteroaryl-S(O)—, and heterocyclyl-S(O)—, where alkyl, cycloalkyl, aryl, heteroaryl and heterocyclyl are as described herein.

“Sulfonyl” refers to groups H—S(O)₂—, alkyl-S(O)₂—, cycloalkyl-S(O)₂—, aryl-S(O)₂—, heteroaryl-S(O)₂—, and heterocyclyl-S(O)₂—, where alkyl, cycloalkyl, aryl, heteroaryl and heterocyclyl are as described herein.

“Sulfinylamino” refers to groups H—S(O)—NR″—, alkyl-S(O)—NR″—, cycloalkyl-S(O)—NR″—, aryl-S(O)—NR″—, heteroaryl-S(O)—NR″—, and heterocyclyl-S(O)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Sulfonylamino” refers to groups H—S(O)₂—NR″—, alkyl-S(O)₂—NR″—, cycloalkyl-S(O)₂—NR″—, aryl-S(O)₂—NR″—, heteroaryl-S(O)₂—NR″—, and heterocyclyl-S(O)₂—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxysulfinylamino” refers to groups HO—S(O)—NR″—, alkylO—S(O)—NR″—, cycloalkylO—S(O)—NR″—, arylO—S(O)—NR″—, heteroarylO—S(O)—NR″—, and heterocyclylO—S(O)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxysulfonylamino” refers to groups HO—S(O)₂—NR″—, alkylO—S(O)₂—NR″—, cycloalkylO—S(O)₂—NR″—, arylO—S(O)₂—NR″—, heteroarylO—S(O)₂—NR″—, and heterocyclylO—S(O)₂—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminothioacyl” refers to groups R″R″N—C(S)—, where each R″ is independently hydrogen, alkyl, cycloalkyl, aryl, heteroaryl, and heterocyclic and where each of alkyl, cycloalkyl, aryl, heteroaryl and heterocyclyl is as described herein.

“Thioacylamino” refers to groups H—C(S)—NR″—, alkyl-C(S)—NR″—, cycloalkyl-C(S)—NR″—, aryl-C(S)—NR″—, heteroaryl-C(S)—NR″—, and heterocyclyl-C(S)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminosulfinyl” refers to groups R″R″N—S(O)—, where each R″ is independently hydrogen, alkyl, cycloalkyl, aryl, heteroaryl, and heterocyclic and where each of alkyl, cycloalkyl, aryl, heteroaryl and heterocyclyl is as described herein.

“Aminosulfonyl” refers to groups R″R″N—S(O)₂—, where each R″ is independently hydrogen, alkyl, cycloalkyl, aryl, heteroaryl, and heterocyclic and where each of alkyl, cycloalkyl, aryl, heteroaryl and heterocyclyl is as described herein.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroaryloxy, heterocyclyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like. For instance, an “optionally substituted amino” group may include amino acid and peptide residues.

In certain embodiments, the term “optionally substituted” is taken to mean that the groups may be substituted from 1 to 3 times independently selected from the groups consisting of oxo/hydroxy, halogen (in particular Cl, Br, F), C₁₋₆ alkyl, C₁₋₆ alkoxy, C₂₋₆ alkenyl, C₂₋₆ alkynyl, C₁₋₆ haloalkyl (in particular —CF₃), C₁₋₆ haloalkoxy (such as —OCF₃), C₂₋₆ alkenyloxy, C₂₋₆ alkynyloxy, arylalkyl (wherein alkyl is C₁₋₆), arylalkoxy (wherein alkyl is C₁₋₆), aryl, cyano, nitro, heteroaryl, C₁₋₆ heteroarylalkyl (wherein alkyl is C₁₋₆), heteroaryloxy, heterocyclyl, heterocyclylalkyl (wherein alkyl is C₁₋₆), heterocyclyloxy, oxyacyl, trialkylsilyl, trifluoromethanethio, trifluoroethenyl, amino, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl.

In other embodiments, the term “optionally substituted” is taken to mean that the groups may be substituted from 1 to 3 times independently selected from the groups consisting of hydroxy, halogen (in particular Cl, Br, F), C₁₋₆ alkyl, C₁₋₆ alkoxy, C₂₋₆ alkenyl, C₁₋₆ haloalkyl (in particular —CF₃), C₁₋₆ haloalkoxy (such as —OCF₃), arylalkyl (wherein alkyl is C₁₋₆), arylalkoxy (wherein alkyl is C₁₋₆), aryl, cyano, nitro, heteroaryl, trialkylsilyl, amino, mono- and di-alkylamino, mono- and di-(substituted alkyl)amino, and mono- and di-arylamino.

In still further embodiments, the term “optionally substituted” is taken to mean that the groups may be substituted from 1 to 3 times independently selected from the groups consisting of hydroxy, halogen (in particular Cl, Br, F), hydroxethyl, hydroxpropyl, methyl, methoxy, cyano, pyridinyl, pyridinylmethyl, pyrazinyl, methylphenyl, benzyl, trimethylsilyl, phenyl, methylpyrazoyl, dimethylamino, fluorophenyl, tert-butyloxycarbonyl, amino or morpholinyl.

The salts of the compounds of the invention are preferably pharmaceutically acceptable, but it will be appreciated that non-pharmaceutically acceptable salts also fall within the scope of the present invention, since these are useful as intermediates in the preparation of pharmaceutically acceptable salts.

The pharmaceutically acceptable salts include acid addition salts, base addition salts, and the salts of quaternary amines and pyridiniums. The acid addition salts are formed from a compound of the invention and a pharmaceutically acceptable inorganic or organic acid including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicylic, sulfamic, or tartaric acids. The counter ion of quatemary amines and pyridiniums include chloride, bromide, iodide, sulfate, phosphate, methansulfonate, citrate, acetate, malonate, fumarate, sulfamate, and tartrate. The base addition salts include but are not limited to salts such as sodium, potassium, calcium, lithium, magnesium, ammonium and alkylammonium. Also, basic nitrogen-containing groups may be quaternised with such agents as lower alkyl halides, such as methyl, ethyl, propyl, and butyl chlorides, bromides and iodides; dialkyl sulfates like dimethyl and diethyl sulfate; and others. The salts may be made in a known manner, for example by treating the compound with an appropriate acid or base in the presence of a suitable solvent.

The compounds of the invention may be in crystalline form and/or as solvates (e.g. hydrates) and it is intended that both forms be within the scope of the present invention. The term “solvate” is a complex of variable stoichiometry formed by a solute (in this invention, a compound of the invention) and a solvent. Such solvents should not interfere with the biological activity of the solute. Solvents may be, not limited to, and by way of example, water, ethanol or acetic acid. Methods of solvation are generally known within the art.

It will be appreciated that the compounds of the invention may have at least one asymmetric centre, and therefore are capable of existing in more than one stereoisomeric form. The invention extends to each of these forms individually and to mixtures thereof, including racemates. The isomers may be separated conventionally by chromatographic methods or using a resolving agent. Alternatively, the individual isomers may be prepared by asymmetric synthesis using chiral intermediates.

In another aspect of the invention, there is provided a pharmaceutical composition that comprises a therapeutically effective amount of one or more of the aforementioned compounds or pharmaceutically acceptable salts thereof, including pharmaceutically acceptable derivatives thereof, and optionally a pharmaceutically acceptable carrier or diluent.

The term “composition” is intended to include the formulation of an active ingredient with encapsulating material as carrier, to give a capsule in which the active ingredient (with or without other carrier) is surrounded by carriers. For instance, one of the preferred formulation forms is an enterically coated tablet form so that the active is released into the small intestine.

The pharmaceutical compositions or formulations include those suitable for oral, rectal, nasal, topical (including buccal and sub-lingual), vaginal, intrathecal or parenteral (including intramuscular, sub-cutaneous and intravenous) administration or in a form suitable for administration by inhalation or insufflation.

The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to one hundred (100) milligrams, per tablet, are accordingly suitable representative unit dosage forms.

The compounds of the present invention can be administered in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

In powders, the carrier is a finely divided solid that is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized moulds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, eg. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds according to the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 5 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The invention also includes the compounds in the absence of carrier where the compounds are in unit dosage form.

The amount of the compound of the invention to be administered may be in the range from about 10 mg to 2000 mg per day, depending on the activity of the compound and the disease to be treated.

Liquids or powders for intranasal administration, tablets or capsules for oral administration and liquids for intravenous administration are the preferred compositions.

The pharmaceutical preparations of the compounds according to the present invention may be co-administered with one or more other active agents in combination therapy. For example, the pharmaceutical preparation of the active compound may be co-administered (for example, separately, concurrently or sequentially), with one or more other agents used to treat pain.

The compounds of the invention have been shown to be beneficial in treating disorders associated with oxidative stress and in particular disorders which would benefit from targeting NFE2L2; NRF2. In this regard, the inventors have focused on the transcription factor nuclear factor erythroid 2-related factor 2 (NFE2L2; NRF2). Under physiological conditions, NFE2L2 is sequestered in the cytosol by Kelch-like ECH associated-protein 1 (Keap1) and ubiquitinated for degradation. However, oxidants and electrophiles trigger release of NFE2L2 from Keap1, translocation to the nucleus, and binding to the antioxidant response element that initiates transcription of >200 antioxidant-related genes. Thus, NFE2L2 was considered to be an attractive therapeutic target to stimulate endogenous production of the multiple antioxidants required to simultaneously detoxify a range of reactive oxygen species. Here, the inventors evaluated the therapeutic actions of dimethyl fumarate (DMF or Tecfidera) and examples of the compounds described in the invention in a mouse spared nerve injury (SNI) model of neuropathic pain. It was observed that these compounds had the ability to reverse, for instance, neuropathic pain behaviours, activate NFE2L2, and resolve mechanistic pathways that maintain neuropathic pain and other types of chronic pain. The upregulation of the NRF2 pathway is observed in oxidatively stressed cells. Accordingly, modulation of the pathway would be beneficial for those conditions associated with oxidative stress such as autoimmune diseases, atherosclerosis, neurodegenerative diseases (including AD and PD), chronic pain, in particular those related to inflammatory pain, infertility, aging and metabolic disorders, all have evidence supporting their precipitation and propagation by ROS/RNS overproduction.

For example, neuropathic (nerve) pain is caused by damage, injury or dysfunction of nerves due to trauma, surgery, disease or chemotherapy. It is often described as burning, painful, cold or akin to electric shocks and may manifest with tingling, pins and needles, numbness or itching. Neuropathic pain can be the primary symptom of a particular condition or disease state, such as cancer, complex regional pain syndrome or post herpetic neuralgia. It can also be associated with other medical conditions or other forms of pain, including pelvic pain, fibromyalgia and orofacial pain. Phantom pain following a limb amputation is also a type of neuropathic pain.

It is also contemplated that the term neuropathic pain also encompasses “peripheral neuropathic pain” as well as central neuropathic pain which is generally defined as pain arising as a direct or indirect consequence of a lesion or disease affecting the peripheral somatosensory system. Peripheral neuropathic pain includes all types of peripheral neuropathic pain, caused by for instance peripheral diabetic neuropathy type 1 or 2, induced by various noxious substances such as alcohol, caused by various deficiencies such as vitamin B1, B6 and/or B12 deficiency, various intoxications, such as hypervitaminosis B6, caused by hypothyroidism, chemotherapy induced polyneuropathy (CIPN) (due to chemotherapeutic agents such as: alkylating agents, such as cis-platinum(II)-diaminedichloride (platinol or cisplatin); oxaliplatin (Eloxatin or Oxaliplatin Medac); and carboplatin (Paraplatin); antitumour antibiotics, including those selected from the group comprising anthracyclines, such as doxorubicin (Adriamycin, Rubex); antimetabolites, including folic acid analogues such as pyrimidine analogues such as 5-fluorouracil (Fluoruracil, 5-FU), gemcitabine (Gemzar), or histone deacetylase inhibitors (HDI) for instance, Vorinostat (rINN); natural alkaloids, including paclitaxel (Taxol); inhibitors of protein tyrosine kinases and/or serine/threonine kinases including Sorafenib (Nexavar), Erlotinib (Tarceva), Dasatanib (BMS-354825 or Sprycel)), drug-induced neuropathy, some compounds for the treatment of infectious diseases (e.g. streptomycin, didanosine or zalcitabine), or other other physiologically toxic compounds. Other peripheral neuropathies that can cause peripheral neuropathic pain include: small fiber neuropathy (SFN), hereditary motor and sensory neuropathies (HMSN), chronic inflammatory demyelinating polyneuropathy (CIDP), trigeminal neuralgia, post-herpetic neuralgia, intercostal neuralgia, entrapment neuropathies (e.g. carpal tunnel syndrome, tarsal tunnel syndrome, abdominal cutaneous nerve entrapment syndrome), sciatic pain, chronic idiopathic axonal polyneuropathy (CIAP), vulvodynia, proctodynia, neuropathy due to infectious disease conditions, such as post-polio syndrome, AIDS or HIV-associated, lyme associated, Sjogren-associated, lymphomatous neuropathy, myelomatous neuropathy, carcinomatous neuropathy, vasculitic/ischaemic neuropathy and other mono- and polyneuropathies.

In an embodiment the invention contemplates the treatment of neuropathic pain associated with chemotherapy—often a side-effect when treating solid tumors. Examples of solid tumors include adrenocortical carcinoma, anal tumor/cancer, bladder tumor/cancer, bone tumor/cancer (such as osteosarcoma), brain tumor, breast tumor/cancer, carcinoid tumor, carcinoma, cervical tumor/cancer, colon tumor/cancer, endometrial tumor/cancer, esophageal tumor/cancer, extrahepatic bile duct tumor/cancer, Ewing family of tumors, extracranial germ cell tumor, eye tumor/cancer, gallbladder tumor/cancer, gastric tumor/cancer, germ cell tumor, gestational trophoblastic tumor, head and neck tumor/cancer, hypopharyngeal tumor/cancer, islet cell carcinoma, kidney tumor/cancer, laryngeal tumor/cancer, leiomyosarcoma, leukemia, lip and oral cavity tumor/cancer, liver tumor/cancer (such as hepatocellular carcinoma), lung tumor/cancer, lymphoma, malignant mesothelioma, Merkel cell carcinoma, mycosis fungoides, myelodysplastic syndrome, myeloproliferative disorders, nasopharyngeal tumor/cancer, neuroblastoma, oral tumor/cancer, oropharyngeal tumor/cancer, osteosarcoma, ovarian epithelial tumor/cancer, ovarian germ cell tumor, pancreatic tumor/cancer, paranasal sinus and nasal cavity tumor/cancer, parathyroid tumor/cancer, penile tumor/cancer, pituitary tumor/cancer, plasma cell neoplasm, prostate tumor/cancer, rhabdomyosarcoma, rectal tumor/cancer, renal cell tumor/cancer, transitional cell tumor/cancer of the renal pelvis and ureter, salivary gland tumor/cancer, Sezary syndrome, skin tumors (such as cutaneous t-cell lymphoma, Kaposi's sarcoma, mast cell tumor, and melanoma), small intestine tumor/cancer, soft tissue sarcoma, stomach tumor/cancer, testicular tumor/cancer, thymoma, thyroid tumor/cancer, urethral tumor/cancer, uterine tumor/cancer, vaginal tumor/cancer, vulvar tumor/cancer, and Wilms' tumor. In one embodiment, the pain is associated with treating the following cancers: bladder cancer, breast cancer, colon cancer, gastroenterological cancer, kidney cancer, lung cancer, including non-small cell lung cancer, ovarian cancer, pancreatic cancer, prostate cancer, proximal or distal bile duct cancer, or melanoma.

In another embodiment the present invention contemplates the treatment of neuropathic pain associated with pain complications arising from an infection such as a bacterial, fungal or viral infection. Examples include shingles, HIV/AIDS, etc.

In still a further embodiment the present invention contemplates the treatment of neuropathic pain associated with back pain, rheumatoid arthritis, trigeminal neuralgia, or diabetic neuropathy.

In another further embodiment the present invention contemplates the treatment of neuropathic pain caused by nerve compression (trapped nerve). Examples include carpal tunnel syndrome or sciatica.

In another further embodiment, the present invention contemplates the treatment of central neuropathic pain associated with stroke or spinal cord injury.

For certain of the abovementioned conditions, it is clear that the compounds may be used prophylactically as well as for the alleviation of symptoms. Thus, references herein to “treatment” or the like are to be understood to include such prophylactic treatment, as well as therapeutic treatments.

Compounds of the invention may be prepared according to the following general schemes A-O below:

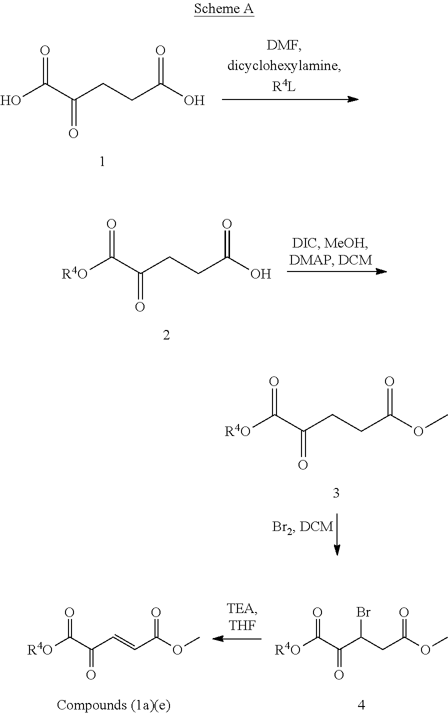

Alpha-keto ester compounds of the invention can be prepared via the synthetic procedures as depicted in Scheme A. Alpha-keto esters 2 can be synthesized from alpha-ketoglutaric acid 1 and a substituted alkyl halide (R⁴L where L represents any leaving group, in this case a halide) as described in Chem. Pharm. Bull. 47(9) 1284-1287 (1999). Subsequent methylation of the carboxylic acid of 2 can be achieved through the use of methanol under Steglich esterification conditions to give ester 3. It will be appreciated by those skilled in the art that the formation of a methyl ester from a carboxylic acid may be achieved through alternative conditions such as reaction with diazomethane or conversion of the carboxylic acid to an acid chloride and coupling with methanol. Bromination of 3 to give bromide 4 can be achieved through treatment with bromine in a suitable solvent, in this case, dichloromethane (DCM). Compounds of formula (1a)(e) can be generated by elimination of the bromide through the use of an amine base, in this case triethylamine (TEA).

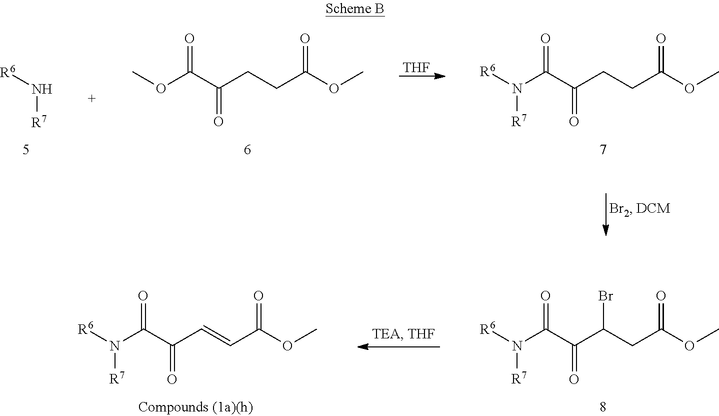

Alpha-keto amide compounds of formula (1a)(h) can be prepared via the synthetic procedures as depicted in Scheme B. Alpha-keto amides 7 can be synthesized from dimethyl 2-oxoglatarate 6 and amine 5 allowed to react in a suitable solvent, as described in J. Org. Chem., 77, 8294-8302 (2012). Bromination of 7 to give bromide 8 can be achieved through treatment with bromine in a suitable solvent, in this case, DCM. Compounds of formula (1a)(h) can be generated by elimination of the bromide through the use of an amine base, in this case TEA.

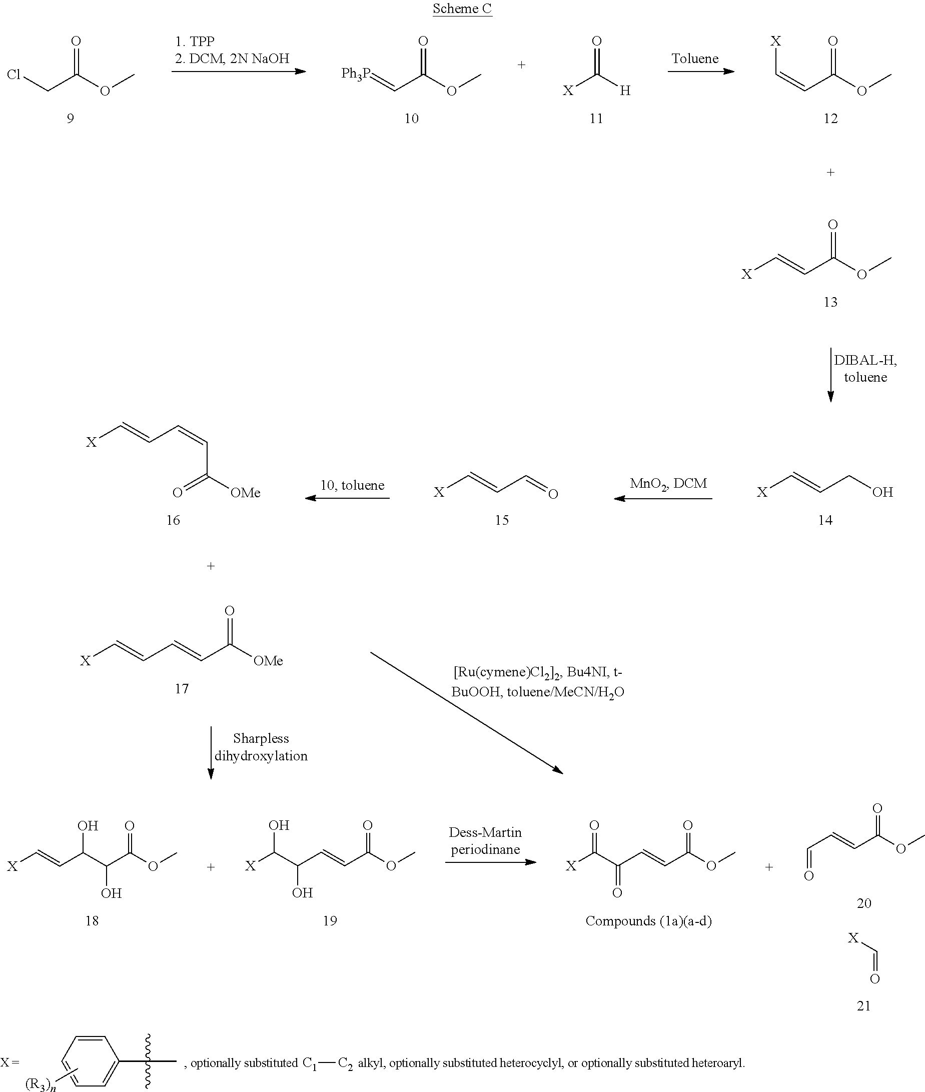

1,2-Diketone compounds of formula (1a)(a-d) can be prepared via the synthetic procedures as depicted in Scheme C. Ylide 10 can be obtained from commercial sources or synthesized from methyl chloroacetate 9 and triphenylphosphine at 90° C., followed by treatment of the solid phosphonium salt product with aqueous sodium hydroxide. Utilizing standard Wittig reaction conditions stabilized ylide 10 can react with aldehydes 11 to generate a separable mixture of cis/trans olefin products 12 and 13. Alternatively, Homer-Wadsworth-Emmons reaction chemistry could be used to access olefin 13, through the use of phosphonate carbanions. It will be appreciated by those skilled in the art that Wittig or Homer-Wadsworth-Emmons olefination reactions can occur under a wide range of conditions and in a wide range of solvents such as toluene or water for example. Separation of the resultant cis/trans olefin products 12 and 13 can be achieved through a variety of chromatographic methods, such as column chromatography, preparative thin layer chromatography and preparative HPLC. Selective reduction of the pure olefin 13 to give allyl alcohol 14 can be achieved through the treatment of pure olefin 13 with DIBAL in an appropriate solvent, in this case toluene. Oxidation of allylic alcohols 14 to generate substituted acrolein derivatives 15 is accomplished through treatment with activated manganese dioxide in an appropriate solvent, in this case DCM. Those skilled in the art will understand that this oxidation could be affected by alternative methods such as Swern oxidation or oxidation with pyridinium dichromate or Dess-Martin periodinane or 2-iodoxybenzoic acid (IBX). Many substituted acrolein derivatives 15 can be obtained from commercial suppliers. Substituted acrolein derivatives 15 can be converted into trans, trans 1,3-butadiene derivatives 17 employing standard Wittig reaction conditions with stabilized ylide 10. Alternatively, Horner-Wadsworth-Emmons reaction chemistry could be used to access the trans, trans 1,3-butadiene derivatives 17, through the use of phosphonate carbanions. Separation of the resultant cis/trans olefin products 16 and 17 can be achieved through a variety of chromatographic methods, such as column chromatography, preparative thin layer chromatography and preparative HPLC. Dihydroxylation of the pure trans, trans 1,3-butadiene derivative 17, can be achieved employing conditions developed by K. Barry Sharpless as described in Chem. Eur. J., 11, 4667-4677 (2005). Those skilled in the art will understand that this dihydroxylation could be affected by alternative methods such as treatment with osmium tetroxide or alkaline potassium permanganate. Separation of the resultant mixture of 1,2-diols 18 and 19 can be achieved through a variety of chromatographic methods, such as column chromatography, preparative thin layer chromatography and preparative HPLC. Reduction of the pure 1,2-diol 19 to give 1,2-diketone compounds of formula (1a)(a-d), can be achieved through treatment with Dess-Martin periodinane in the absence of solvent. This technique limits the production of aldehyde by-products from the alternate oxidative cleavage reaction pathway. Care must be taken as the reaction can initiate rapidly and is exothermic.

An alternative synthesis of the cis/trans olefin products 16 and 17 could be achieved through the Wittig reaction of stabilised ylide methyl (2E)-4-(triphenylphosphoranylidene)-2-butenoate and aldehyde 11.

An alternative synthesis of 1,2-diketone compounds of formula (1a)(a-d) could be to directly generate the 1,2-diketone moiety through ruthenium-catalyzed oxidation of the diene 17 as outlined in Org. Lett., 13, 2274-2277 (2011).

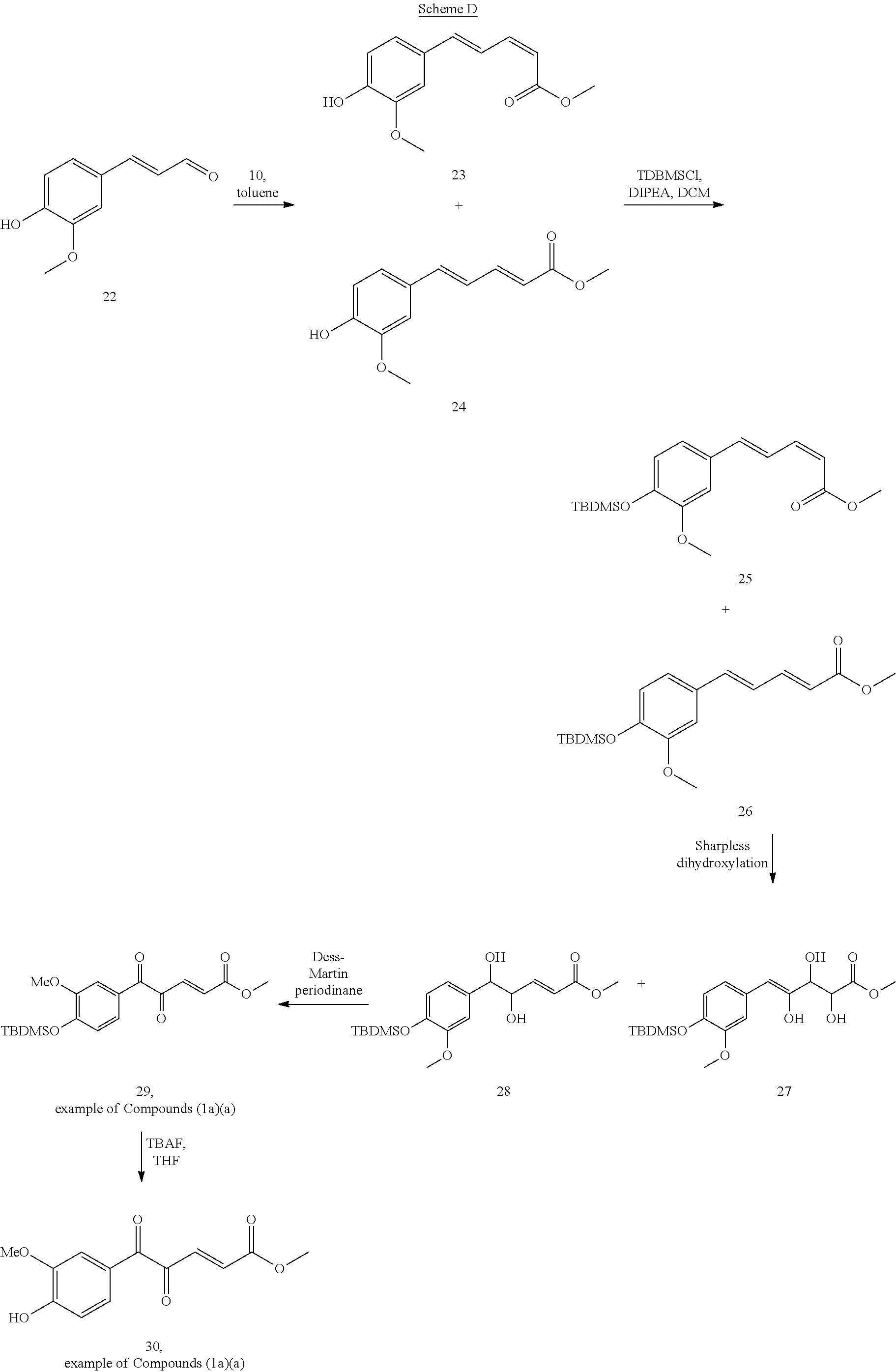

Scheme D outlines the synthesis of aromatic 1,2-diketones that contain a phenol functional group as this group requires a protection/deprotection strategy. This is demonstrated with the specific example of 4-hydroxy-3-methoxycinnamaldehyde (ferulaldehyde/coniferyl aldehyde). Aldehyde 22 can be converted to a mixture of dienes 23 and 24 via a Wittig reaction with stabilized ylide 10, as described above. The inseparable mixture of dienes 23 and 24 can be converted to a mixture of protected phenols 25 and 26, through treatment with TBDMSCl and base in a suitable solvent, in this case DIPEA and DCM respectively. Pure trans, trans 1,3-butadiene 26, can be obtained via trituration of the crystalline mixture of 25 and 26 with hexane. Other derivatives could potentially be separated through a variety of chromatographic methods, such as column chromatography, preparative thin layer chromatography and preparative HPLC. Compounds of formula (1a)(a) can be synthesized as described above via dihydroxylation and diol oxidation. Removal of the silane protecting group can be achieved using TBAF in a suitable solvent, in this case THF, to generate 30 from 29. Silane deprotection could also be achieved through treatment with other fluoride salts such as KF. Conceivably other protecting groups could be used to protect the phenol as described in Greene's Protective Groups in Organic Synthesis, Fifth Edition by Wuts P G M, New Jersey, John Wiley & Sons, Inc. 2014.

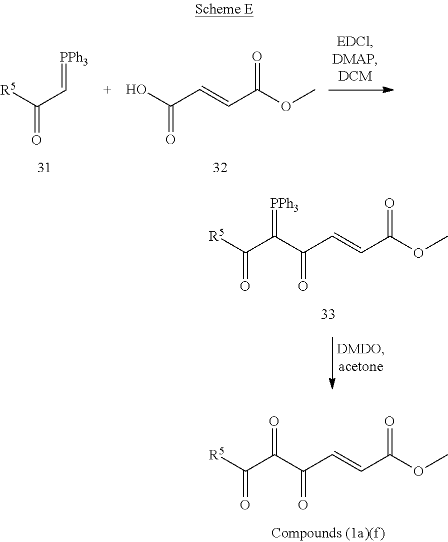

1,2,3-tricarbonyl compounds of formula (1a)(f) can be prepared via the synthetic procedures as depicted in Scheme E. Stabilized phosphorus ylides 31 can react with monomethyl fumarate 32 employing N-(3-dimethylaminopropyl)-N′-ethylcarbodiimide hydrochloride (EDC) coupling conditions as outlined in J. Org. Chem., 60, 8231-8235 (1995) to give compounds of formula (1a)(f). Those skilled in the art would understand that couplings of this nature could occur through other methods of activating the carboxylic acid group. These could include conversion to an acid chloride, conversion to an NHS ester or the use of other coupling agents such as N,N′-dicyclohexylcarbodiimide. Ylide 33 can be converted to tricarbonyl systems through oxidation of the carbon phosphorus bond using distilled dimethyl dioxirane (DMDO) in acetone as outlined in J. Org. Chem., 60, 8231-8235 (1995).

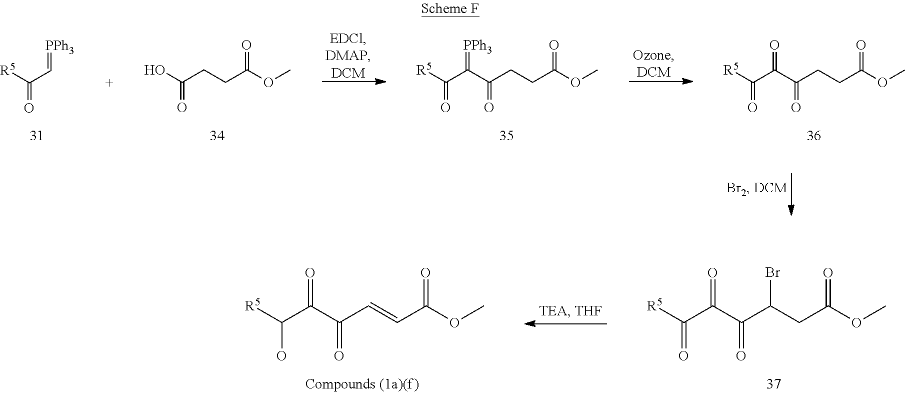

An alternative route is outlined in Scheme F, which is to couple stabilized phosphorus ylides 31 to mono-methyl hydrogen succinate 34 to give unsaturated ylide 35 as described above. Ylide 35 can then be converted to tricarbonyl compound 36 through oxidation with ozone in an appropriate solvent, in this case DCM. Other oxidants for ylide oxidation could be used such as singlet oxygen or DMDO. Bromination of 36 to give bromide 37 can be achieved through treatment with bromine in a suitable solvent, in this case, DCM. Compounds of formula (1a)(f) can be generated by elimination of the bromide 37 through the use of an amine base, in this case TEA. In the case where R⁵ creates an ester derivative 38, selective hydrolysis using, for example phosphate buffer at pH 7.4, would result in the formation of carboxylic acid derivative 39, an example of compounds of formula (1a)(f), Scheme G.

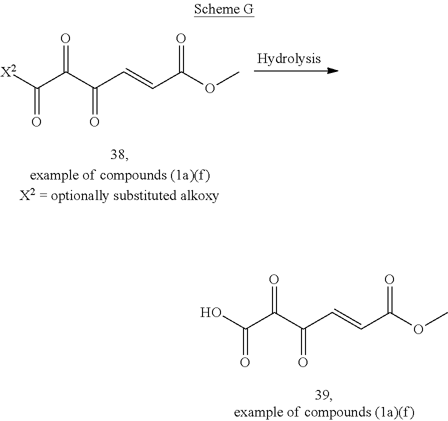

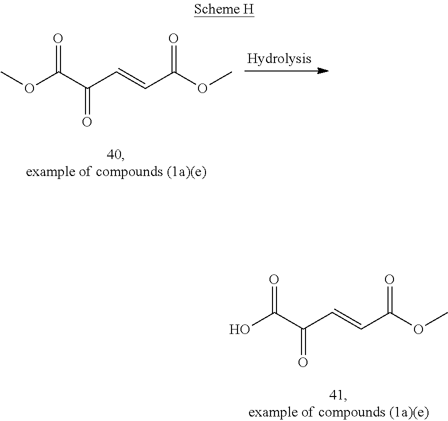

Compounds of formula (1a)(e) such as 1,5-dimethyl (2E)-4-oxopent-2-enedioate (40), can be hydrolysed, using for example phosphate buffer at pH 7.4, to give alpha-keto acid 41, an example of compounds of formula (1a)(e), Scheme H.

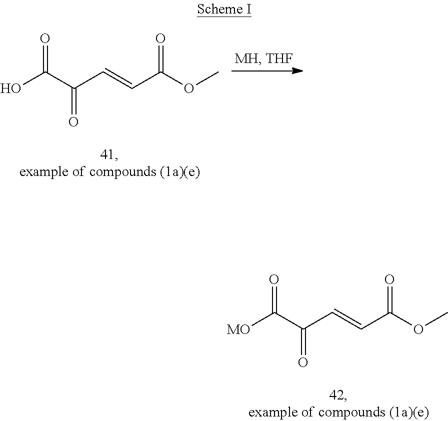

Salts of alpha-keto acid 41 can be synthesized through reaction with suitable metal hydride (MH) such a sodium hydride in THF utilizing the methodology outlined in for instance WO2015/172083, Scheme I. Those skilled in the art will appreciate that other salts of carboxylic acids could include those of other group I (alkali) metals such as potassium or lithium or group II (alkaline earth) metals such as magnesium or calcium. These can be obtained through reaction with appropriate metal hydrides or metal carbonates in appropriate solvents. Carboxylic acid salts of other metals such as silver can be obtained similarly by reaction with silver carbonate. Amine bases such as triethylamine can also be used to generate salts of carboxylic acids.

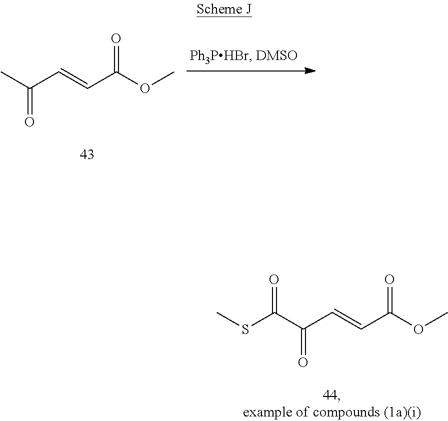

Alpha-keto thioester 44, an example of compounds of formula (1a)(i), can be synthesized under oxidative bromination and Kornblum oxidation conditions using triphenylphosphine hydrobromide and DMSO from methyl (2E)-4-oxopent-2-enoate (43) utilizing the methodology outlined in Chem. Eur. J. 20, 662-667 (2014), Scheme J.

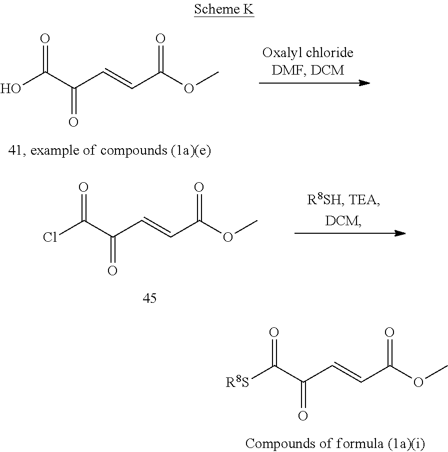

More broadly alpha-keto thioester compounds of formula (1a)(i) can be prepared via the synthetic procedures depicted in Scheme K. Alpha-keto acid chloride 45 can be synthesized from (3E)-5-methoxy-2,5-dioxopent-3-enoic acid (41) by treatment with oxalyl chloride in DCM or using thionyl chloride in an appropriate solvent. Alpha-keto thioester compounds of formula (1a)(i) can then be synthesized by treating the acid chloride 45 with a thiol in the presence of TEA in an appropriate solvent, in this case DCM, as outlined in Adv. Synth. Catal., 358, 3212-3230 (2016).

This methodology would also allow access to alpha-keto ester and alpha-keto amide compounds of formula (1a)(e) and (h) through the reaction of alcohols and amines with acid chloride 45, in an appropriate solvent such as diethyl ether and base such as TEA, Scheme L.

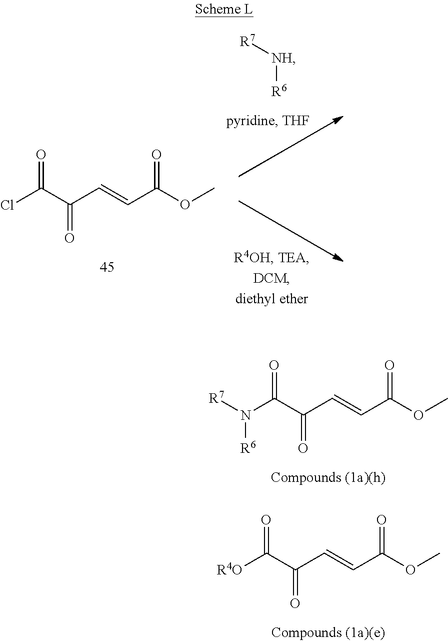

Access to alpha-keto ester compounds of formula (1a)(e) can be achieved through selective transesterification of the alpha-keto ester 1,5-dimethyl (2E)-4-oxopent-2-enedioate (40) as outlined in Scheme M. Those skilled in the art will recognize that transesterifications of esters can be facilitated by catalysts such as N-heterocyclic olefins as detailed in Org. Lett. 2016, 18, 2208-2211.

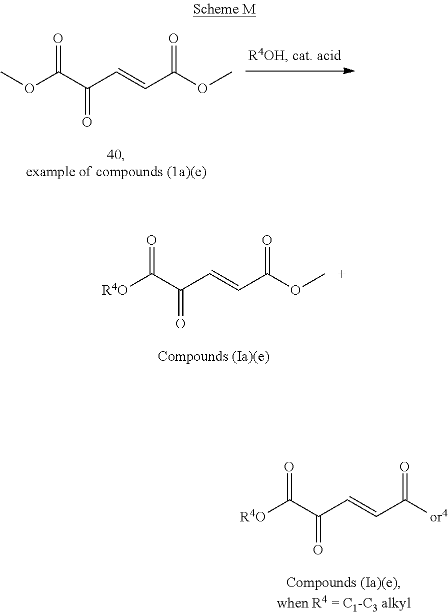

Compounds of formula (1a)(i) such as methyl (2E)-5-(methylsulfanyl)-4,5-dioxopent-2-enoate (44), can be hydrolysed to give alpha-keto acid 41, an example of compounds of formula (1a)(e), Scheme N.

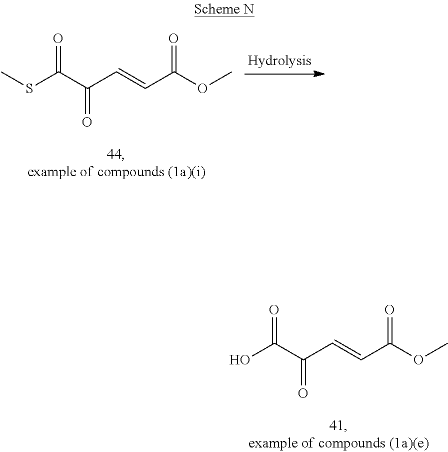

Steglich esterification conditions could be utilized to esterify alpha-keto acid (3E)-5-methoxy-2,5-dioxopent-3-enoic acid (41) to access compounds of formula (1a)(e). Those skilled in the art will know that numerous alternative coupling reagents and conditions could be used to couple alpha-keto acid 41 with an alcohol to yield alpha-keto ester compounds of formula (1a)(e). These include but are not limited to N-(3-dimethylaminopropyl)-N-ethylcarbodiimide hydrochloride (EDAC), (1-[bis(dimethylamino)methylene]-1H-1,2,3-triazolo[4,5-b]pyridinium 3-oxide hexafluorophosphate (HATU) and propylphosphonic anhydride (T3P).

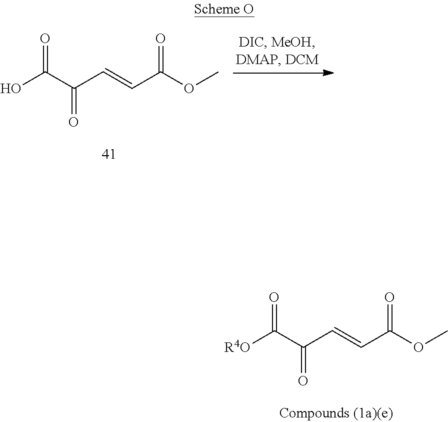

Another variation is to add, remove or modify the substituents of the product to form new derivatives. This could be achieved again by using standard techniques for functional group interconversion, well known in the industry such as those described in Comprehensive Organic Transformations: A Guide to Functional Group Preparations by Larock R C, New York, VCH Publishers, Inc. 1989.

In a preferred embodiment the invention provides a method of treating disorders associate with oxidative stress including the step of administering to a subject in need thereof a compound of (Ia):

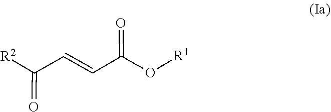

or pharmaceutically acceptable salts thereof:
where R¹ is C₁-C₃ alkyl and
where R² is selected from:
e)

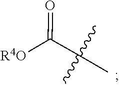

wherein R⁴ is selected from H, optionally substituted alkyl, optionally substituted aryl, optionally substituted heteroaryl, and optionally substituted heterocyclyl.

In relation the above embodiment, R⁴ may be selected from is H, C1-C8 substituted alkyl, optionally substituted aryl, optionally substituted heteroaryl, and optionally substituted heterocyclyl.

In other embodiments, R⁴ is selected from H and C1-C8 alkyl.

In other embodiments, R⁴ is selected from H and C1-C3 alkyl.

In another embodiment, R⁴ is H or C1-C2 alkyl.

In another embodiment, R⁴ is C1-C2 alkyl.

In another embodiment, R⁴ is C1 alkyl.

In another embodiment, R⁴ is C2 alkyl.

In another embodiment, R⁴ is H.

Without wishing to be bound by theory, the in vivo results from the present inventors have demonstrated disease state amelioration through site directed target engagement of a specific biochemical pathway (NRF2) in oxidatively stressed tissue using ROS/RNS activated release of MMF from compounds of formula (1a)(e). From ¹H and ¹³C NMR studies the inventors believe that alpha-keto acid, (3E)-5-methoxy-2,5-dioxopent-3-enoic acid (41, Example J) may be the systemically circulating 1,2-dicarbonyl compound delivering MMF to the oxidatively stressed tissue in vivo.

Dimethyl fumrarate (Tecfidera®) and diroximel fumarate (Vumerity™) are orally administered to patients and almost entirely converted to monomethyl fumarate (MMF) in the small intestine through esterase mediated and spontaneous hydrolysis. MMF has limited reactivity with glutathione in its anionic form and does not react with CYP enzymes, passing from the small intestine through the liver and into the bloodstream where it is delivered systemically as the therapeutic agent. Limited esterase hydrolysis in the liver converts some MMF to fumaric acid which integrates into the tricarboxylic acid cycle and is ultimately expelled in the breath as carbon dioxide. Systemically delivered MMF is similarly metabolized, initiated by esterases in other cells and tissues.

The inventors utilized a method of oral administration with compound in vehicle being gavage into the stomach of mice were selective spontaneous hydrolysis of the alpha-keto ester moiety in the stomach and/or the small intestine may provide the corresponding alpha-keto acid. Selective hydrolysis is believed to come from alpha-keto esters being much more susceptible to spontaneous hydrolysis than similar carboxylic esters. The alpha-keto acid is thought to be the species absorbed into the pre-systemic blood supply from the small intestine.

Fumarate based drugs are often administered in enteric coated formulations to avoid exposure to the stomach acid which could lead to the formation of fumaric acid. This breakdown to fumaric acid is also unwanted since fumaric acid has no therapeutic effect. In certain embodiments the compounds of the present invention (and in particular those of formula (1a)(e)) are intended to be delivered orally with an enteric coated form which is intended to deliver the compounds to the small intestine. In the small intestine it is anticipated that selective spontaneous and esterase mediated hydrolysis of the alpha-keto ester moiety to give the corresponding alpha-keto acid would occur. Again, the alpha-keto acid is expected to be the species absorbed into the pre-systemic blood supply from the small intestine.

Molecular modeling of alpha-keto acid (Example J), Table 1, shows it will be significantly less electrophilic (the more negative w^{q} is, the more electrophilic that carbon is) in its anionic form than the parent alpha-keto ester (Example A), and MMF, particularly as it will be almost entirely ionized at the near neutral to basic pH of the small intestine and neutral pH of the blood and liver. We estimate the pKₐ of alpha-keto acid (Example J) at around 1.56±0.54 (ACD Labs prediction—Scifinder) and this is supported by empirical measurements of other related alpha-keto acids (J. Pharm. Sci. 2016, 105(2), 664-672). This means the equilibrium at pH 7.4 is 794,328:1 in favour of being ionised, contrasting this is the equilibrium of MMF (measured pKa=3.63, Arch Dermatol Res. 2010, 302(7), 531-8) of 5,888:1 in favour of being ionised.

TABLE 1
Calculated relative reactivity of MMF,
Ex A and Ex J with nucleophiles

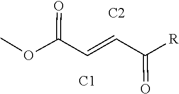

| Compound | C1 w^{q} (eV) | C2 w^{q} (eV) |
|---|---|---|
| MMF | −0.697703809 | −0.677091582 |
| MMF⁻ | −0.015324169 | −0.042815045 |
| Ex A | −0.916406033 | −0.294186407 |
| Ex J | −0.967321393 | −0.299389213 |
| Ex J⁻ | −0.0005984 | −0.000858403 |

Relative reactivity towards nucleophiles will also be dependent on aspects of the equilibrium of the 1,2-dicarbonyl and its geminal diol in an aqueous environment at a particular pH. At near neutral pH the alpha-keto acid is predominantly deprotonated and in the keto form (4:1 ketoacid:gem diol) as measured by NMR.

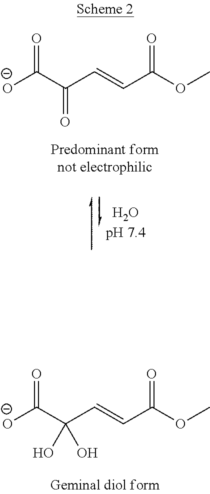

Once absorbed through the small intestine and as a consequence of the predominance of the charged non-electrophilic form, it is unlikely alpha-keto acid will form conjugates with glutathione as it moves through pre-systemic circulation from the small intestine to the liver. In the liver we expect CYP interactions of Ex J to be minimal based on the finding that alpha-keto acids are not efficient iron chelators (Eur Food Res Technol 2016, 242, 179-188) and that MMF does not interact with CYPs and is not a CYP inducer. Some esterase mediated hydrolysis of Ex J to generate the corresponding diacid would probably occur in the liver due to the broader range of ester substrates they accept. The diacid may undergo further metabolism through oxidative decarboxylation to fumaric acid and carbon dioxide.

The ultimate result of first pass metabolic processes on alpha-keto ester Ex A, and other compounds of formula (1a)(e), is thought to be systemic delivery of alpha-keto acid Ex J into the blood stream in amounts high enough to effectively deliver a therapeutic dose of MMF to specifically to cells and tissues under oxidative stress.

Without wishing to be bound by any particular theory the inventors are of the opinion that metabolite Ex J is likely the 1,2-dicarbonyl compound that is eliciting the targeted delivery of MMF to tissue under oxidative stress to restore cellular homeostasis. The incredibly low electrophilicity (predicted by the modeling of the inventors) of Ex J in its charged state, which predominates at physiological pH of tissue and blood, they think is key to this targeting, allowing Ex J to remain inactive until activated by hydrogen peroxide or peroxynitrite oxidative cleavage to MMF. This is also believed to be key to reducing on- and off-target side-effects via reduced systemic activation of the NRF2 pathway and limited interaction with reactive amines and thiols of other proteins. Conversion of Ex A to Ex J is thought to occur in the stomach and/or the small intestine.

alpha-Keto acid Ex J, once systemically delivered is believed to elicit its site selective therapeutic effects by localized cleavage to MMF in the presence of oxidative stress metabolites hydrogen peroxide and peroxynitrite. Therapeutic action of MMF includes activation of the NRF2 pathway to engage cellular antioxidant, anti-inflammatory and detoxification functions, activation of the hydroxycarboxylic acid 2 (HCA2) receptor (also known as GPR109A in humans), which exerts its effect through downstream inhibition of NFκB activation via activation of inhibitor, protein deacetylase sirtuin-1 (SIRT1) and other anti-inflammatory actions independent of NRF2 and HCA2 including prevention of the release of the proinflammatory cytokine interleukin-1β by succinating active thiols of gasdermin-D. Non-succinated gasdermin-D normally forms a membrane pore that allows extracellular release of interleukin-1β.

In order that the present invention may be more readily understood, we provide the following non-limiting examples.

### SYNTHETIC EXAMPLES

All anhydrous solvents were commercially obtained and stored in Sure-Seal bottles under nitrogen or transferred to an Inert Corporation Solvent Purification System and dispensed from there. All other reagents and solvents were purchased as the highest grade required and used without further purification. All organic extracts were dried over anhydrous magnesium sulfate (MgSO₄). Thin-layer chromatography (TLC) used aluminum sheets coated with silica gel 60 F₂₅₄ from Merck and were visualised using ultraviolet light. Melting points were taken on a Reichert Thermovar Kofler apparatus and are uncorrected. Infrared spectra were recorded on a Perkin Elmer Spectrum 400 FT-IR/FT-FIR Spectrometer as neat samples unless otherwise stated. ¹H NMR and ¹³C NMR, spectra were acquired on an Agilent 500 MHz spectrometer. High resolution mass spectrometry (HRMS) was performed on an Agilent 6230 ESI-TOF LCMS. All yields reported refer to isolated material judged to be homogeneous by TLC and NMR spectroscopy.

In the examples below, in case the structures contain one or more stereogenic cetres, the respective structure is depicted in an arbitrary configuration. There structures depict single enantiomers as well as mixtures of enantiomers in all ratios and/or mixtures of diastereomers in all ratios.

General Procedures

General Procedure A: Synthesis of Alpha-Keto Esters

A mixture of alpha-ketoglutaric acid (2 equiv.) and dicyclohexylamine (1 equiv.) was dissolved at 50° C. in anhydrous DMF (40 ml/6.8 mmol of alpha-ketoglutaric acid) under an atmosphere of nitrogen. Alkyl halide (1 equiv.) was then added and the mixture stirred at 50° C. until complete. Once complete the reaction mixture was poured into water and partitioned with diethyl ether. The organic layer was separated, and the aqueous layer further extracted with diethyl ether. The organics were then combined and extracted with brine, dried (MgSO₄), filtered and concentrated in vacuo to give the crude product which was used without further purification.

General Procedure B: Methyl Ester Formation

To a solution of carboxylic acid (1 equiv.) in anhydrous DCM (5 ml/1 mmol of carboxylic acid) under an inert atmosphere was added DIC (1.1 equiv.) and MeOH (3 equiv.) followed by DMAP (0.1 equiv.). The mixture was stirred at ambient temperature until complete. Once complete the mixture was diluted with diethyl ether and water was added. The aqueous layer was extracted with diethyl ether (2×) and the combined organics then washed with water and brine, dried (MgSO₄), filtered and concentrated in vacuo to give the crude product. The crude product was purified by column chromatography.

General Procedure C: Bromination of Alpha-Ketoglutaric Acid Derivatives

To a solution of alpha-ketoglutaric acid derivative (1.0 equiv.) dissolved in anhydrous DCM (15 ml/1 mmol of alpha-ketoglutaric acid derivative) was added bromine (1.5 equiv.) dropwise. The mixture was stirred at 35° C. under an inert atmosphere until complete. Once complete, the volatiles were removed in vacuo and the crude product was used without further purification.

General Procedure D: Elimination of HBr

Triethylamine (2 equiv.) was added to a solution of bromide (1 equiv.) in anhydrous THF (10 ml/1 mmol of bromide) and the mixture stirred under an inert atmosphere, protected from light at ambient temperature until complete. Once complete the volatiles were removed in vacuo and the crude residue purified by column chromatography.

General Procedure E: Synthesis of Alpha-Keto Amides

To a solution of dimethyl 2-oxoglutarate (1.0 equiv.) in anhydrous THF (2.5 ml/1.5 mmol dimethyl 2-oxoglutarate) was added amine (1.5 equiv.) and the mixture stirred at ambient temperature under an inert atmosphere until complete. Once complete the volatiles were removed in vacuo and the crude residue purified by column chromatography.

General Procedure F: Wittig Reaction

A suspension of aldehyde (1 equiv.) and stabilized ylide (1.1 equiv.) in an appropriate solvent (1 ml/1 mmol. of aldehyde) was placed in a pressure vessel equipped with a magnetic stir bar. The vial was sealed and placed in an oil bath at 150° C. for 10 min. The reaction mixture was cooled and then transferred to a round-bottom flask and the volatiles removed in vacuo. Hexane or a mixture of 10% ethyl acetate in hexane was added and the mixture stirred for 10 minutes then filtered through Celite® to remove the majority of the triphenylphosphine oxide by-product. The filtrate was concentrated in vacuo the crude residue purified by column chromatography.

General Procedure G: Ester Reduction to Alcohol

A solution of ester (1 equiv.) in anhydrous toluene (10 ml/2.2 mmol of ester) at 0° C. was added DIBAL-H (1.0 M in toluene, 2 equiv.) dropwise and the mixture stirred at 0° C. under an inert atmosphere until complete. Once complete the reaction mixture was diluted with ethyl acetate and a saturated solution of Rochelle's salt was added dropwise until no more gas was evolved. The solid material formed was removed by filtration through Celite®. The filtrate was then washed with water and the aqueous layer further extracted with ethyl acetate. The combined organics were dried (MgSO₄), filtered and concentrated in vacuo to give the crude product. The crude product was purified by column chromatography.

General Procedure H: Oxidation of an Alcohol to Aldehyde

Activated manganese dioxide (10 equiv.) was added to a solution of alcohol (1 equiv.) in anhydrous DCM (3 ml/1 mmol. of alcohol) at ambient temperature and left to stir under an inert atmosphere until complete. Once complete DCM was added to the reaction mixture and the solid material removed by filtration through Celite®. The volatiles were then removed under reduced pressure to give the desired aldehyde which was used without further purification.

General Procedure I: Sharpless Dihydroxylation

To a 1:1 solution of t-BuOH and water (8 ml/1 mmol. of olefin) was added AD-mix β (1.4 g/1 mmol. of olefin) and the mixture was stirred until two clear phases were visible with the lower phase bright yellow. To this mixture was added a solution of methanesulfonamide (1 equiv.) and olefin (1 equiv.) dissolved in a 1:1 solution of t-BuOH and water (2 ml/1 mmol. of olefin), note the olefin may require heating to dissolve. The reaction mixture was then stirred at ambient temperature until complete. Some olefins require the reaction mixture to be heated to between 30° C. and 50° C. to solubilize them. Once complete sodium sulfite (1.5 g/1 mmol of olefin) was added, and the mixture stirred at ambient temperature for 30 minutes. Diethyl ether (10 ml/1 mmol olefin) was added to the reaction mixture, and after separation of the layers, the aqueous phase was further extracted with the organic diethyl ether (3×5 ml/1 mmol olefin). The combined organic extracts were dried (MgSO₄), filtered and concentrated in vacuo to give the crude diol which was purified by column chromatography.

General Procedure J: Diol Oxidation

To diol (1 equiv.) that was cooled in an ice bath was added the Dess-Martin periodinane (2.1 equiv.) and the mixture stirred with a glass stirring rod until complete. Once complete the crude residue was purified by column chromatography.

General Procedure K: Transesterification of Alpha-Keto Esters

To a solution of Example A (1 equiv.) in alcohol was added conc. sulfuric acid and the mixture stirred at ambient temperature overnight under an inert atmosphere until complete. Once complete the volatiles were removed in vacuo and the crude residue purified by column chromatography.

### Intermediate A: 5-(benzyloxy)-4,5-dioxopentanoic acid

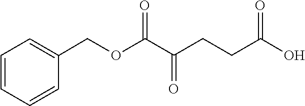 [SMILES: O=C(O)CCC(=O)C(=O)OCc1ccccc1]

alpha-Ketoglutaric acid was reacted as described in General Procedure A to give the title compound (956 mg, 65% yield) as a light blue semi-solid. Characterisation data matched literature reported values Chem. Pharm. Bull., 47, 1284-1287 (1999).

### Intermediate B: 1-benzyl 5-methyl 2-oxopentanedioate

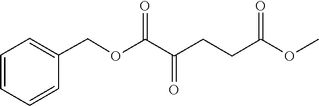 [SMILES: COC(=O)CCC(=O)C(=O)OCc1ccccc1]

Intermediate A was reacted as described in General Procedure B to give the title compound (108 mg, 41% yield) as a colourless oil. R_{f}=0.38 (20% EA in hexane); ¹H NMR (500 MHz, CDCl₃) δ 7.45-7.32 (m, 5H), 5.29 (s, 2H), 3.68 (s, 3H), 3.17 (t, J=6.8 Hz, 2H), 2.67 (t, J=6.5 Hz, 1H); ¹³C NMR (125 MHz, CDCl₃) δ 192.35, 172.51, 160.41, 134.54, 128.98, 128.86, 128.81, 68.30, 52.17, 34.45, 27.54; IR (neat) 2955, 1728, 1606, 1588, 1499, 1456, 1438 cm⁻¹.

### Intermediate C: 1-benzyl 5-methyl 3-bromo-2-oxopentanedioate

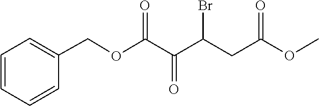 [SMILES: COC(=O)CC(Br)C(=O)C(=O)OCc1ccccc1]

Intermediate B was reacted as described in General Procedure C to give the title compound as an orange oil, which was used without further purification.

### Intermediate D: methyl 4-(benzylcarbamoyl)-4-oxobutanoate

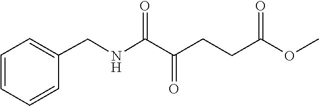 [SMILES: COC(=O)CCC(=O)C(=O)NCc1ccccc1]

Dimethyl 2-oxoglutarate was reacted with benzyl amine as described in General Procedure E to give the title compound (251 mg, 70% yield) as a colourless oil. Characterisation data matched literature reported values J. Org. Chem., 77, 8294-8302 (2012).

### Intermediate E: methyl 4-(benzylcarbamoyl)-3-bromo-4-oxobutanoate

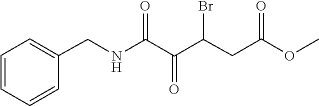 [SMILES: COC(=O)CC(Br)C(=O)C(=O)NCc1ccccc1]

Intermediate D was reacted as described in General Procedure C to give the title compound as an orange oil, which was used without further purification.

### Intermediate F: methyl (triphenylphosphoranylidene)acetate

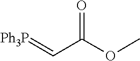

Methyl chloroacetate (4 g, 36.9 mmol) and triphenyl phosphine (8.7 g, 33.2 mmol) were combined and stirred with heating to 90° C. under a nitrogen atmosphere. Once a glass formed the glass was broken up, crushed and washed with toluene to remove any unreacted starting material. The phosphonium salt was then dissolved in DCM and to this solution was added an aqueous 2 N NaOH solution (38.7 ml), which was stirred for 1 h at room temperature. The reaction mixture was then separated, and the organic layer washed with water and brine, dried (MgSO₄) and concentrated in vacuo to afford the title compound (9.45 g, 77% yield). Characterisation data matched literature reported values J. Org Chemin. 79, 1467-1472 (2014).

### Intermediate G: methyl (2E,4E)-5-phenylpenta-2,4-dienoate

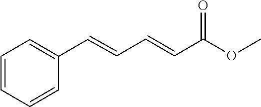

trans-Cinnamaldehyde was reacted with Intermediate F in toluene as described in General Procedure F to give the title compound (1.541 g, 82% yield) as a colourless solid. Characterisation data matched literature reported values Eur. J. Org. Chem., 5204-5213 (2017).

### Intermediate H: methyl (2E)-4,5-dihydroxy-5-phenylpent-2-enoate

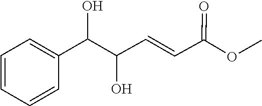 [SMILES: COC(=O)/C=C/C(O)C(O)c1ccccc1]

Intermediate G was reacted as described in General Procedure I to give the title compound (403 mg, 34% yield) as a colourless oil. R_{f}=0.5 (50% EA in hexane); ¹H NMR (CDCl₃, 500 MHz) δ 7.37-7.31 (m, 5H), 6.74 (dd, J=16.0, 4.0 Hz, 1H), 6.09 (dd, J=16.0, 1.5 Hz, 1H), 4.55-4.53 (m, 1H), 4.41 (brs, 1H), 3.70 (s, 3H), 2.84 (d, J=4.0 Hz, 1H), 2.78 (d, J=2.5 Hz, 1H); ¹³C NMR (CDCl₃, 125 MHz) δ 166.6, 145.6, 139.6, 128.7, 128.6, 126.8, 122.0, 77.0, 75.3, 51.6; IR (neat) 3425, 1705, 1660, 1495, 1449, 1437, 1391, 1311, 1277 cm⁻¹.

### Intermediate I: methyl (2E,4E)-5-(4-hydroxy-3-methoxyphenyl)penta-2,4-dienoate

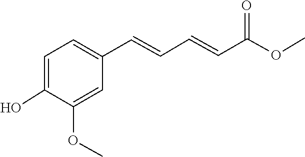 [SMILES: COC(=O)/C=C/C=C/c1ccc(O)c(OC)c1]

4-Hydroxy-3-methoxycinnamaldehyde was reacted with Intermediate F in toluene as described in General Procedure F to give the title compound as an inseparable mixture with cis isomer, methyl (2Z,4E)-5-(4-hydroxy-3-methoxyphenyl)penta-2,4-dienoate (315 mg).

methyl (2Z,4E)-5-(4-hydroxy-3-methoxyphenyl)penta-2,4-dienoate: R_{f}=0.43 (30% EA in hexane); ¹H NMR (500 MHz, Chloroform-d) δ 8.00 (ddd, J=15.6, 11.4, 1.1 Hz, 1H), 7.07 (d, J=1.9 Hz, 1H), 7.02 (dd, J=8.2, 2.0 Hz, 1H), 6.89 (d, J=8.2 Hz, 1H), 6.75 (d, J=15.8 Hz, 1H), 6.73 (d, J=11.3 Hz, 1H), 5.76 (s, 1H), 5.68 (d, J=11.2 Hz, 1H), 3.94 (s, 3H), 3.76 (s, 3H).

Intermediate I: R_{f}=0.37 (30% EA in hexane); ¹H NMR (500 MHz, Chloroform-d) δ 7.44 (dd, J=15.2, 10.9 Hz, 1H), 7.02-6.96 (m, 2H), 6.90 (d, J=8.1 Hz, 1H), 6.83 (d, J=15.5 Hz, 1H), 6.73 (dd, J=15.5, 10.8 Hz, 1H), 5.95 (d, J=15.2 Hz, 1H), 5.75 (s, 1H), 3.94 (s, 3H), 3.77 (s, 3H).

### Intermediate J: methyl (2E,4E)-5-{4-[(tert-butyldimethylsilyl)oxy]-3-methoxyphenyl}penta-2,4-dienoate

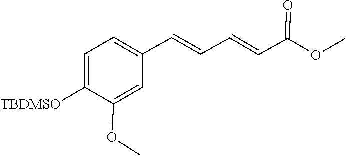 [SMILES: COC(=O)/C=C/C=C/c1ccc(O[Si](C)(C)C(C)(C)C)c(OC)c1]

To a mixture of Intermediate I and methyl (2Z,4E)-5-(4-hydroxy-3-methoxyphenyl)penta-2,4-dienoate (315 mg, 1.67 mmol) in anhydrous DCM (2 ml) was added in DIPEA (872 μl, 5.01 mmol) followed by t-butyldimethylsilyl chloride (503 mg, 3.34 mmol). The mixture was stirred at ambient temperature under an inert atmosphere for 7.5 hours. The crude reaction mix was partitioned in water and twice extracted with DCM. The combined organics were washed with water and brine, dried (MgSO₄) and concentrated in vacuo to give the crude product which was purified by trituration with hexane to give the title compound (165 mg, 28% yield) as a yellow crystalline solid. ¹H NMR (500 MHz, Chloroform-d) δ 7.44 (dd, J=15.2, 10.8 Hz, 1H), 6.98-6.92 (m, 2H), 6.87-6.80 (m, 2H), 6.78-6.69 (m, 1H), 5.95 (d, J=15.2 Hz, 1H), 3.84 (s, 3H), 3.77 (s, 3H), 0.99 (s, 9H), 0.16 (s, 6H); ¹³C NMR (125 MHz, Chloroform-d) δ 167.80, 151.33, 146.65, 145.35, 140.86, 130.13, 124.48, 121.26, 121.16, 119.77, 110.38, 77.41, 77.16, 76.91, 55.58, 51.59, 25.80, 18.62, −4.48; IR (neat) 3033, 2931, 2887, 2858, 1707, 1624, 1590, 1566, 1507, 1473, 1459, 1420, 1386, 1352 cm⁻¹; HRMS (ESI): calculated for C₁₉H₂₉O₄Si 349.1835 [M+H]⁺, found 349.1843.

### Intermediate K: methyl (2E)-5-{4-[(tert-butyldimethylsilyl)oxy]-3-methoxyphenyl}-4,5-dihydroxypent-2-enoate

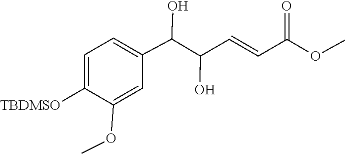 [SMILES: COC(=O)/C=C/C(O)C(O)c1ccc(O[Si](C)(C)C(C)(C)C)c(OC)c1]

Intermediate J was reacted as described in General Procedure I, with heating to 30° C., to give the title compound (113 mg, 42% yield) as a colourless oil. R_{f}=0.4 (40% EA in hexane); ¹H NMR (500 MHz, Chloroform-d) δ 6.85-6.82 (m, 2H), 6.78 (dd, J=8.1, 2.0 Hz, 1H), 6.74 (dd, J=15.7, 4.4 Hz, 1H), 6.07 (dd, J=15.7, 1.8 Hz, 1H), 4.48 (d, J=7.1 Hz, 1H), 4.39 (ddd, J=6.8, 4.6, 1.8 Hz, 1H), 3.80 (s, 3H), 3.70 (s, 3H), 2.67 (s, 1H), 2.55 (s, 1H), 0.99 (s, 9H), 0.15 (s, 6H). ¹³C NMR (125 MHz, Chloroform-d) δ 166.79, 151.26, 145.94, 145.36, 133.09, 121.92, 121.07, 119.38, 110.65, 77.16, 75.54, 55.67, 51.77, 25.83, 18.57, −4.50; IR (neat) 3420, 2959, 2930, 2887, 2858, 1725, 1707, 1660, 1585, 1514, 1464, 1436, 1419, 1391 cm⁻¹. HRMS (ESI): calculated for C₁₉H₃₀NaO₆Si 405.1709 [M+Na]⁺, found 405.1703.

### Intermediate L: methyl (2E)-3-(4-fluorophenyl)prop-2-enoate

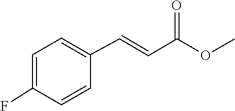

4-fluorobenzaldehyde was reacted with Intermediate F in toluene as described in General Procedure F, to give the title compound (1.005 g, 40% yield) as a colourless solid. Characterisation data matched literature reported values J. Org. Chem., 69, 4216-4226 (2004).

### Intermediate M: (2E)-3-(4-fluorophenyl)prop-2-en-1-ol

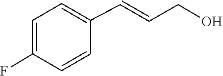 [SMILES: OC/C=C/c1ccc(F)cc1]

Intermediate L was reacted as described in General Procedure G, to give the title compound (619 mg, 62% yield) as a colourless solid. Characterisation data matched literature reported values J. Am. Chem. Soc., 135, 12690-12693 (2013).

### Intermediate N: (2E)-3-(4-fluorophenyl)prop-2-enal

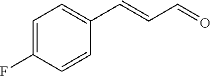 [SMILES: O=C/C=C/c1ccc(F)cc1]

Intermediate M was reacted as described in General Procedure H, to give the title compound (394 mg, 94% yield) as a colourless solid. Characterisation data matched literature reported values Org. Lett., 13, 992-994 (2011).

### Intermediate O: methyl (2E,4E)-5-(4-fluorophenyl)penta-2,4-dienoate

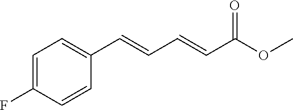

Intermediate N was reacted with Intermediate F in toluene as described in General Procedure F, to give the title compound (95 mg, 24% yield) as a colourless solid. Characterisation data matched literature reported values Org. Chem. Front., 6, 796-800 (2019).

### Intermediate P: methyl (2E)-5-(4-fluorophenyl)-4,5-dihydroxypent-2-enoate

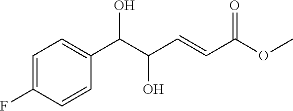 [SMILES: COC(=O)/C=C/C(O)C(O)c1ccc(F)cc1]

Intermediate O was reacted as described in General Procedure I, to give the title compound (21 mg, 19% yield) as a colourless solid. R_{f}=0.36 (50% ethyl acetate in hexane); ¹H NMR (500 MHz, Chloroform-d) δ 7.36-7.30 (m, 2H), 7.11-7.03 (m, 2H), 6.73 (dd, J=15.7, 4.5 Hz, 1H), 6.09 (dd, J=15.7, 1.8 Hz, 1H), 4.56 (d, J=6.9 Hz, 1H), 4.43-4.35 (m, 1H), 3.72 (s, 3H), 2.62 (s, 2H); ¹³C NMR (125 MHz, Chloroform-d) δ 166.59, 162.92 (d, J=247.0 Hz), 145.30, 135.45 (d, J=3.3 Hz), 128.68 (d, J=8.1 Hz), 122.51, 115.82 (d, J=21.5 Hz), 76.61, 75.56, 51.84; IR (neat) 3459, 3275, 2958, 1712, 1663, 1608, 1511, 1474, 1431, 1414, 1373; Mp 105-107° C.

### Intermediate Q: methyl (2E,4E)-5-(4-methoxyphenyl)penta-2,4-dienoate

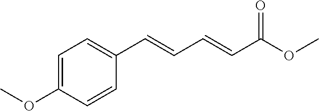

(2E)-3-(4-methoxyphenyl)prop-2-enal was reacted with Intermediate F in toluene as described in General Procedure F, to give the title compound (139 mg, 21% yield) as a colourless solid. Characterisation data matched literature reported values Org. Chem. Front., 6, 796-800 (2019).

### Intermediate R: methyl (2E)-4,5-dihydroxy-5-(4-methoxyphenyl)pent-2-enoate

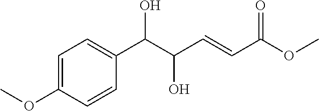

Intermediate Q was reacted as described in General Procedure I, to give the title compound (40 mg, 28% yield) as a yellow oil. R_{f}=0.23 (50% ethyl acetate in hexane); ¹H NMR (500 MHz, Chloroform-d) δ 7.32-7.23 (m, 2H), 6.93-6.87 (m, 2H), 6.73 (dd, J=15.7, 4.3 Hz, 1H), 6.10 (dd, J=15.7, 1.8 Hz, 1H), 4.50 (d, J=7.1 Hz, 1H), 4.40 (ddd, J=6.7, 4.4, 1.8 Hz, 1H), 3.81 (s, 3H), 3.71 (s, 3H), 2.71 (s, 1H), 2.51 (s, 1H); ¹³C NMR (125 MHz, CDCl₃) δ 166.75, 159.96, 145.71, 131.73, 128.18, 122.11, 114.32, 76.92, 75.54, 55.47, 51.77; IR (neat) 3416, 3003, 2953, 2902, 2839, 1709, 1659, 1611, 1585, 1513, 1459, 1437, 1391 cm⁻¹.

### Intermediate S: methyl 4,5-dioxo-5-(piperidin-1-yl)pentanoate

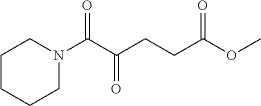 [SMILES: COC(=O)CCC(=O)C(=O)N1CCCCC1]

Dimethyl 2-oxoglutarate was reacted with piperidine as described in General Procedure E to give the title compound (44 mg, 13% yield) as a colourless oil. R_{f}=0.26 (30% ethyl acetate in hexane); ¹H NMR (500 MHz, Chloroform-d) δ 3.68 (s, 3H), 3.58-3.52 (m, 2H), 3.41-3.36 (m, 2H), 3.07-3.01 (m, 2H), 2.71-2.65 (m, 2H), 1.71-1.64 (m, 2H), 1.64-1.57 (m, 5H); ¹³C NMR (125 MHz, Chloroform-d) δ 199.72, 173.06, 165.46, 52.03, 46.86, 42.62, 34.84, 27.13, 26.50, 25.51, 24.53; IR (neat) 2941, 2860, 1736, 1715, 1634, 1446, 1440, 1369, 1350, 1321 cm⁻¹; HRMS (ESI): calculated for C₁₁H₁₈NO₄ 228.1236 [M+H]⁺, found 228.1232.

### Example A: 1,5-dimethyl (2E)-4-oxopent-2-enedioate

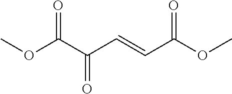

Dimethyl 2-oxoglutarate was reacted as described in General Procedure C, without heating, followed by reaction of the product bromide as described in General Procedure D, to give the title compound (1.999 g, 80% yield). Characterisation data matched authentic sample purchased from commercial source.

### Example B: 5-benzyl 1-methyl (2E)-4-oxopent-2-enedioate

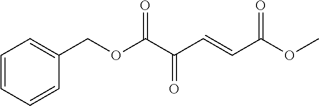 [SMILES: COC(=O)/C=C/C(=O)C(=O)OCc1ccccc1]

Intermediate C was reacted as described in General Procedure D to give the title compound (11 mg, 12% yield) as a yellow oil. R_{f}=0.35 (DCM); ¹H NMR (500 MHz, CDCl₃) δ 7.60 (d, J=16.0 Hz, 1H), 7.47-7.30 (m, 5H), 6.95 (d, J=16.1 Hz, 1H), 5.34 (s, 2H), 3.83 (s, 3H); ¹³C NMR (125 MHz, CDCl₃) δ 182.42, 165.24, 160.59, 135.56, 134.32, 134.29, 129.13, 128.94, 128.92, 68.61, 52.72; IR (neat) 3086, 3067, 3037, 2959, 1734, 1722, 1698, 1637, 1499, 1456, 1439, 1384 cm⁻¹; HRMS (ESI): calculated for C₁₃H₁₂O₅ 249.0763 [M+H]⁺, found 249.0769; Mp 45-47° C.

### Example C: methyl (2E)-4-(benzylcarbamoyl)-4-oxobut-2-enoate

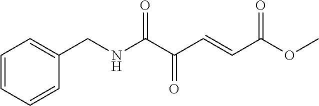 [SMILES: COC(=O)/C=C/C(=O)C(=O)NCc1ccccc1]

Intermediate E was reacted as described in General Procedure D to give the title compound (173 mg, 73% yield) as a yellow solid. Characterisation data matched literature reported values J. Org. Chem., 77, 8294-8302 (2012).

### Example D: methyl (2E)-4,5-dioxo-5-phenylpent-2-enoate

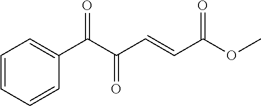 [SMILES: COC(=O)/C=C/C(=O)C(=O)c1ccccc1]

Intermediate H was reacted as described in General Procedure J to give the title compound (318 mg, 71%) as a yellow oil. R_{f}=0.55 (DCM); ¹H NMR (CDCl₃, 500 MHz) δ 8.00 (d, J=8.0 Hz, 1H), 7.68 (t, J=7.5 Hz, 1H), 7.54-7.49 (m, 3H), 6.90 (d, J=16.0 Hz, 1H), 3.85 (s, 3H); ¹³C NMR (CDCl₃, 125 MHz) δ 191.0, 190.9, 165.2, 135.4, 135.3, 135.1, 130.3, 129.0, 52.6; HRMS (ESI): calculated for C₁₂H₁₀O₄ 219.0657 [M+H]⁺, found 219.0648; IR (neat) 3067, 2955, 1727, 1670, 1622, 1596, 1450, 1437, 1310 cm⁻¹.

### Example E: methyl (2E)-5-{4-[(tert-butyldimethylsilyl)oxy]-3-methoxyphenyl}-4,5-dioxopent-2-enoate

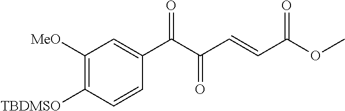 [SMILES: COC(=O)/C=C/C(=O)C(=O)c1ccc(O[Si](C)(C)C(C)(C)C)c(OC)c1]

Intermediate K was reacted as described in General Procedure J to give the title compound (23 mg, 74% yield) as a yellow oil. R_{f}=0.22 (60% DCM in hexane); ¹H NMR (500 MHz, Chloroform-d) δ 7.55 (d, J=2.0 Hz, 1H), 7.50-7.43 (m, 2H), 6.92-6.84 (m, 2H), 3.87 (s, 3H), 3.83 (s, 3H), 1.00 (s, 9H), 0.20 (s, 6H); ¹³C NMR (125 MHz, Chloroform-d) δ 191.68, 190.02, 165.48, 152.50, 151.73, 135.99, 135.11, 126.23, 125.96, 120.82, 112.12, 55.70, 52.70, 25.71, 18.66, −4.39; IR (neat) 2953, 2931, 2858, 1732, 1691, 1661, 1588, 1508, 1464, 1436, 1420 cm⁻¹.

### Example F: methyl (2E)-5-(4-hydroxy-3-methoxyphenyl)-4,5-dioxopent-2-enoate

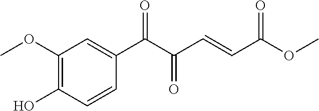

To a solution of Example E (27 mg, 0.07 mmol) in anhydrous THF (1 ml) was added TBAF (19 mg, 0.07 mmol) and the solution stirred at ambient temperature under an inert atmosphere for 6 hours. Ethyl acetate and saturated sodium bicarbonate solution were added to the reaction mixture and the organic layer separated. The aqueous layer was further extracted with ethyl acetate (×3) and the combined organics washed with water and brine. The organics were then dried (MgSO₄), filtered and concentrated in vacuo to give the crude product, which was purified by column chromatography to give the title compound (3 mg, 14% yield) as a yellow solid. R_{f}=0.22 (60% DCM in hexane); Mp 91-94° C.; ¹H NMR (500 MHz, Chloroform-d) δ 7.61-7.54 (m, 2H), 7.47 (dd, J=16.2, 0.8 Hz, 1H), 6.98 (dd, J=8.2, 0.8 Hz, 1H), 6.86 (dd, J=16.2, 0.8 Hz, 1H), 6.26 (s, 1H), 3.98 (s, 3H), 3.83 (s, 3H); ¹³C NMR (125 MHz, Chloroform-d) δ 191.53, 189.77, 165.48, 152.63, 147.19, 135.97, 135.14, 127.30, 125.00, 114.63, 110.96, 56.39, 52.71; IR (neat) 3337, 2920, 2850, 1731, 1672, 1644, 1585, 1513, 1453, 1433, 1381 cm⁻¹.

### Example G: methyl (2E)-5-(4-fluorophenyl)-4,5-dioxopent-2-enoate

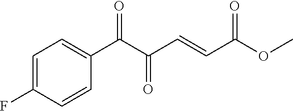 [SMILES: COC(=O)/C=C/C(=O)C(=O)c1ccc(F)cc1]

Intermediate P was reacted as described in General Procedure J to give the title compound (12 mg, 58% yield) as a yellow solid. R_{f}=0.6 (DCM); ¹H NMR (500 MHz, Chloroform-d) δ 8.15-8.01 (m, 2H), 7.54 (d, J=16.1 Hz, 1H), 7.24-7.15 (m, 2H), 6.90 (d, J=16.1 Hz, 1H), 3.84 (s, 3H); ¹³C NMR (125 MHz, Chloroform-d) δ 190.38, 189.10, 167.10 (d, J=258.9 Hz), 165.34, 135.50, 135.28, 133.42 (d, J=10.0 Hz), 128.60 (d, J=2.9 Hz), 116.56 (d, J=21.9 Hz), 52.75; IR (neat) 3083, 3070, 2960, 1720, 1677, 1631, 1595, 1507, 1435, 1412, 1324, 1308 cm⁻¹; Mp 53-54° C.

### Example H: methyl (2E)-5-(4-methoxyphenyl)-4,5-dioxopent-2-enoate

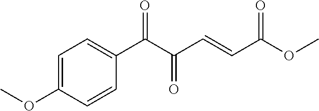

Intermediate R was reacted as described in General Procedure J to give the title compound (20 mg, 50% yield) as a yellow solid. R_{f}=0.44 (DCM); ¹H NMR (500 MHz, Chloroform-d) δ 8.00 (d, J=8.6 Hz, 2H), 7.49 (d, J=16.2 Hz, 1H), 6.98 (d, J=8.6 Hz, 2H), 6.87 (d, J=16.2 Hz, 1H), 3.90 (s, 3H), 3.83 (s, 3H); ¹³C NMR (125 MHz, Chloroform-d) δ 191.47, 189.60, 165.49, 165.37, 135.87, 135.08, 133.00, 125.11, 114.55, 55.82, 52.68; IR (neat) 2953, 2844, 1723, 1688, 1666, 1648, 1592, 1566, 1508, 1458, 1427, 1303 cm⁻¹; HRMS (ESI): calculated for C₁₃H₁₃O₅ 249.0763 [M+H]⁺, found 249.0770; Mp 69-71° C.

### Example I: methyl (2E)-5-(methylsulfanyl)-4,5-dioxopent-2-enoate

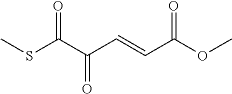

PPh₃.HBr (7.48 g, 21.8 mmol.) was placed in a dry three-neck round bottom flask under nitrogen with condenser. The flask was cooled with a water bath and anhydrous DMSO (60 ml) was added dropwise with stirring. Methyl (2E)-4-oxopent-2-enoate (1.396 g, 10.9 mmol.) was introduced into the reaction mixture after 5 minutes, and the resulting mixture was stirred at 50° C. under an inert atmosphere until complete. After completion of the reaction (˜2.5 h, evaluated by TLC, 20% ethyl acetate in hexane), saturated ammonium chloride solution (300 ml) was added, and the product was extracted with ethyl acetate (1×150 ml and 2×100 ml). The combined organics were washed with water (75 ml) and then brine (3×75 ml), separated and the organics dried (MgSO₄), filtered and concentrated under reduced pressure to give a crude residue. This was purified by silica gel flash column chromatography (squat column eluting with 20% ethyl acetate in hexane) to give the title compound (1.221 g, 59% yield) as a yellow crystalline solid. R_{f}=0.6 (20% ethyl acetate in hexane); ¹H NMR (500 MHz, Chloroform-d) δ 7.68 (d, J=15.9 Hz, 1H), 7.05 (d, J=16.0 Hz, 1H), 3.84 (s, 3H), 2.41 (s, 3H); ¹³C NMR (125 MHz, Chloroform-d) δ 191.52, 183.08, 165.23, 135.95, 132.26, 52.73, 11.56; IR (neat) 3077, 3061, 2957, 2848, 1716, 1688, 1667, 1638, 1437, 1306, 1288, 1205, 1182, 1154 cm⁻¹; Mp 37-42° C.

### Example J: (3E)-5-methoxy-2,5-dioxopent-3-enoic acid

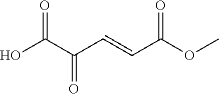

1,5-dimethyl (2E)-4-oxopent-2-enedioate, Example A (200 mg, 1.16 mmol.) was dissolved in 100 mM phosphate buffer at pH 7.4 (20 ml) and stirred overnight at ambient temperature, protected from light. The solution was then diluted with 0.1 M HCl (60 ml) and 1 M HCl was added until solution reached pH 1. The mixture was then extracted with 3:1 chloroform/isopropanol (5×40 ml). Combined organics were dried (MgSO₄), filtered and concentrated in vacuo. The obtained material was then taken up in DCM and filtered again to give the title compound (114 mg, 62% yield) as a yellow solid. ¹H NMR (500 MHz, Chloroform-d) δ 7.75 (d, J=16.0 Hz, 1H), 7.19 (d, J=16.0 Hz, 1H), 6.63 (brs, 1H), 3.86 (s, 3H); ¹³C NMR (125 MHz, Chloroform-d) δ 183.06, 165.05, 159.42, 137.43, 132.38, 52.94; IR (neat) 3536, 3344, 3084, 3067, 2967, 1770, 1732, 1687, 1452, 1440, 1370, 1314, 1263, 1217 cm⁻¹; Mp 44-45° C.

### Example K: (3E)-5-methoxy-2,5-dioxopent-3-enoic acid, sodium salt

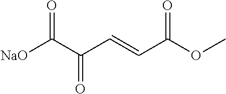

To a stirred solution of (3E)-5-methoxy-2,5-dioxopent-3-enoic acid, Example J (110 mg, 0.70 mmol.) in anhydrous THF (3 ml) at room temperature was added sodium hydride 60% in oil (27 mg, 0.69 mmol.) in one portion. After addition, the mixture was heated to 60° C. for 3 hours under an inert atmosphere, and then cooled to room temperature. The solid precipitate was collected by filtration and washed with anhydrous THF, and further dried in vacuo to give the title compound (84 mg, 67% yield) as a pale-yellow solid. ¹H NMR (500 MHz, DMSO-d6) δ 6.99 (d, J=16.0 Hz, 1H), 6.56 (d, J=16.1 Hz, 1H), 3.73 (s, 4H); ¹³C NMR (125 MHz, DMSO-d6) δ 195.31, 167.05, 165.74, 138.64, 130.61, 52.12; IR (neat) 3102, 1723, 1662, 1624, 1438, 1402, 1300, 1268, 1215, 1193, 1166 cm⁻¹; Mp>190° C.

### Example L: 5-[2-(2,5-dioxopyrrolidin-1-yl)ethyl] 1-methyl (2E)-4-oxopent-2-enedioate

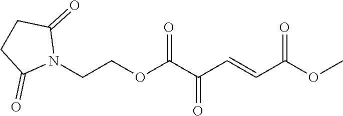 [SMILES: COC(=O)/C=C/C(=O)C(=O)OCCN1C(=O)CCC1=O]

To a solution of (3E)-5-methoxy-2,5-dioxopent-3-enoic acid, Example J (77 mg, 0.49 mmol.) and DMF (1 drop) in anhydrous DCM (5 ml) under an inert atmosphere was added oxalyl chloride (54 μl, 0.63 mmol.), drop-wise at 0° C. and the reaction was protected from light. The solution was then stirred for a further 1 hour at room temperature, after which time the volatiles were then removed in vacuo to give crude acid chloride, which was used without further purification. Crude acid chloride was taken up in anhydrous diethyl ether (5 ml) and DCM (3 ml) and to this was added N-(2-hydroxyethyl)succinimide (70 mg, 0.49 mmol.) followed by triethylamine (67 μl, 0.49 mmol.) at ambient temperature. The reaction mixture was stirred at ambient temperature and monitored for completion by TLC. After 45 min the volatiles were removed in vacuo and the crude mixture purified by column chromatography (70% ethyl acetate in hexane) to give title compound (28 mg, 20% yield) as a pale-yellow oil. R_{f}=0.4 (70% ethyl acetate in hexane); ¹H NMR (500 MHz, Chloroform-d) δ 7.58 (d, J=16.1 Hz, 1H), 6.98 (d, J=16.3 Hz, 1H), 4.48 (t, J=5.2 Hz, 2H), 3.91 (t, J=5.2 Hz, 2H), 3.84 (s, 3H), 2.74 (s, 4H); ¹³C NMR (125 MHz, Chloroform-d) δ 181.89, 177.13, 165.25, 160.35, 135.64, 134.13, 63.33, 52.70, 37.34, 28.25; IR (neat) 2956, 1695, 1625, 1433, 1399, 1367, 1303, 1248, 1186 cm⁻¹; HRMS (ESI): calculated for C₁₂H₁₄NO₇ 284.0770 [M+H]⁺, found 284.0784.

### Example M: methyl (2E)-4,5-dioxo-5-(piperidin-1-yl)pent-2-enoate

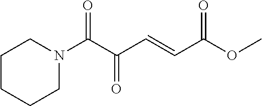 [SMILES: COC(=O)/C=C/C(=O)C(=O)N1CCCCC1]

Intermediate S was reacted as described in General Procedure C to generate its bromide and then reacted as described in General Procedure D to give the title compound (27 mg, 63% yield) as a yellow oil. ¹H NMR (500 MHz, Chloroform-d) δ 7.22 (d, J=16.2 Hz, 1H), 6.79 (d, J=16.2 Hz, 1H), 3.82 (s, 3H), 3.64-3.58 (m, 2H), 3.36-3.30 (m, 2H), 1.74-1.55 (m, 6H). ¹³C NMR (125 MHz, Chloroform-d) δ 189.90, 165.44, 164.02, 136.95, 134.89, 52.68, 47.11, 42.77, 26.49, 25.52, 24.45. IR (neat) 2943, 2860, 1730, 1685, 1635, 1446, 1370, 1309, 1281, 1254, 1180 cm⁻¹; HRMS (ESI): calculated for C₁₁H₁₆NO₄ 226.1079 [M+H]⁺, found 226.1076.

### Example N and O: 5-ethyl 1-methyl (2E)-4-oxopent-2-enedioate and 1,5-diethyl (2E)-4-oxopent-2-enedioate

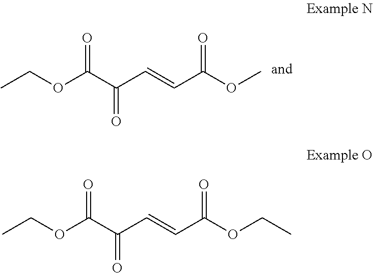

1,5-dimethyl (2E)-4-oxopent-2-enedioate, Example A (25 mg, 0.15 mmol.) was reacted with ethanol (0.25 ml) containing 1 drop of conc. sulfuric acid as described in General Procedure K, to give Example N (10 mg, 37% yield) as a yellow oil. R_{f}=0.54 (20% ethyl acetate in hexane); ¹H NMR (500 MHz, Chloroform-d) δ 7.61 (d, J=16.0 Hz, 1H), 6.96 (d, J=16.0 Hz, 1H), 4.38 (q, J=7.1 Hz, 2H), 3.84 (s, 3H), 1.39 (t, J=7.1 Hz, 3H). ¹³C NMR (125 MHz, Chloroform-d) δ 182.76, 165.30, 160.79, 135.42, 134.39, 63.15, 52.72, 14.13, and Example O (1 mg, 3% yield) as a yellow oil. R_{f}=0.64 (20% ethyl acetate in hexane); ¹H NMR (500 MHz, Chloroform-d) δ 7.60 (d, J=16.0 Hz, 1H), 6.96 (d, J=16.0 Hz, 1H), 4.39 (q, J=7.2 Hz, 2H), 4.29 (q, J=7.1 Hz, 2H), 1.40 (t, J=7.2 Hz, 3H), 1.34 (t, J=7.1 Hz, 3H).

### Example P and Q: 1-methyl 5-propan-2-yl (2E)-4-oxopent-2-enedioate and 1,5-bis(propan-2-yl) (2E)-4-oxopent-2-enedioate

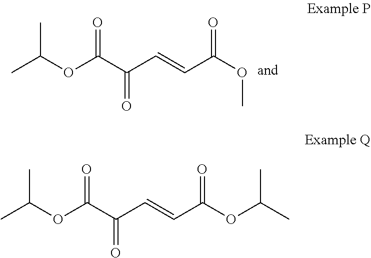

1,5-dimethyl (2E)-4-oxopent-2-enedioate, Example A (50 mg, 0.29 mmol.) was reacted with propan-2-ol (0.5 ml) containing 1 drop of conc. sulfuric acid as described in General Procedure K, to give Example P (19 mg, 33% yield) as a yellow oil. R_{f}=0.44 (20% ethyl acetate in hexane); ¹H NMR (500 MHz, Chloroform-d) δ 7.60 (d, J=16.0 Hz, 1H), 6.94 (d, J=16.0 Hz, 1H), 5.20 (hept, J=6.3 Hz, 1H), 3.84 (s, 3H), 1.37 (d, J=6.3 Hz, 6H). ¹³C NMR (125 MHz, Chloroform-d) δ 183.12, 165.35, 160.44, 135.25, 134.50, 71.59, 52.71, 21.72, and Example Q (4 mg, 6% yield) as a yellow oil. R_{f}=0.57 (20% ethyl acetate in hexane); ¹H NMR (500 MHz, Chloroform-d) δ 7.55 (d, J=16.0 Hz, 1H), 6.92 (d, J=16.0 Hz, 1H), 5.21 (hept, J=6.3 Hz, 1H), 5.13 (hept, J=12.5, 6.3 Hz, 1H), 1.38 (d, J=6.3 Hz, 6H), 1.31 (d, J=6.3 Hz, 6H). ¹³C NMR (125 MHz, cdcl₃) δ 183.36, 164.42, 160.61, 136.45, 134.07, 71.54, 69.64, 21.88, 21.75.

### BIOLOGICAL EXAMPLES

Cell Culture

NRF2/ARE responsive luciferase reporter HEK293 cells (Signosis, Santa Clara, USA) were seeded at 1×10⁵ cells/well in 48-well plates (Corning, Tewksbury, USA) in supplemented Dulbecco's Modified Eagle Medium (DMEM) containing 4.5 g/L glucose (HyClone, Pittsburgh, USA) (10% fetal bovine serum (FBS) (ThermoFisher Scientific, Waltham, USA) and 1% penicillin/streptomycin (Gibco, Waltham, USA)) and cultured overnight at 37° C., 5% CO₂, in a humidified environment. The next morning, the media was replaced with supplemented DMEM (1% FBS (ThermoFisher Scientific) and 1% penicillin/streptomycin (Gibco)). Cells were then treated with a concentration range of each example of compounds described in the invention or monomethyl fumarate (0-100 μM), together with 10 μM hydrogen peroxide (H₂O₂) (Sigma Aldrich, St Louis, USA), 10 μM peroxynitrite (PN) (Sigma Aldrich), or supplemented media (negative control). tert-Butylhydroquinone (tBHQ; 10 μM) (Sigma-Aldrich) was used as positive control. Each condition was run in triplicate and observations confirmed over at least two additional replications.

Luciferase Assay

After 16 h of incubation with examples of compounds described in the invention and ROS/RNS, cells were washed gently with 500 μL PBS (Sigma-Aldrich) and then incubated in 40 μL passive lysis buffer (Promega, Madison, USA) at room temperature. After 20 mins, 30 μL was transferred from each well to a 96-well clear-bottom, white-wall plate (Corning). Luciferase substrate (Signosis; 150 μL) was added to each well and gently mixed. Luminesence was immediately read on a Synergy HTX multi-mode microplate reader (BioTek, Winooski, USA).

Animals

Pathogen-free adult male and female C57BL/6J mice (8 weeks old on arrival; The Jackson Laboratory, Bar Harbor, USA) were used. Mice were housed five per cage in a light- and temperature-controlled room (12:12-h light-dark cycle, light on at 7:00 AM) with food and water available ad libitum. All procedures were approved by the MD Anderson Cancer Center Animal Care and Use Committee.

Spared Nerve Injury (SNI) Surgery

SNI was performed under inhaled isoflurane anesthesia. The tibial and common peroneal nerves were isolated, tightly ligated with 6-0 silk, and transected immediately distal to the ligation. The sural nerve was left intact. Animals were monitored post-operatively until fully ambulatory prior to return to their home cage.

Drug Administrations

Compounds were suspended with methylcellulose (viscosity 15 cP, 2% w/v in water;) and administered by oral gavage. For SNI anti-nociception, compounds were administered for 3 days (350 μmol/kg/day), beginning 7 days after SNI, and continuing for 3 (reflex tests) or 7 days (conditioned place preference tests). For leukopenia assessment, compounds were administered to naïve mice for 10 days (350 μmol/kg/day). Equivolume methylcellulose (2% w/v) was used as vehicle control for all compounds. An independent investigator dosed the mice in order to maintain blinding to treatment groups for the other investigators who performed the behavioral testing.

Tactile Allodynia

Testing was conducted blind with respect to group assignment. Mice received at least three 60-minute habituations to the test environment prior to behavioral testing. Mice were placed in a small plexiglass enclosure on a mesh stand. Tactile allodynia was measured using the von Frey test. The 50% paw withdrawal threshold was determined using the “up-down” method.

Dynamic Allodynia

Testing was conducted immediately after completion of the von Frey test. Dynamic mechanical hypersensitivity was measured by light stroking (velocity is ˜2 cm/s) of the external lateral side of the injured hind paw in the direction from heel to toe with a paintbrush. The paw withdrawal response was scored according to the following criteria, score=0: walking away or occasionally very brief paw lifting (s; 1 s); score=1: sustained lifting (>2 s) of the stimulated paw toward the body; score=2: a strong lateral lifting above the level of the body; score=3: flinching or licking of the affected paw. Average scores for each mouse were obtained from three stimulations at intervals of at least 3 min.

Conditioned Place Preference Assay

Spontaneous pain was tested using a conditioning paradigm with retigabine as the conditioned stimulus to briefly relieve pain. Mice were first allowed to freely explore the conditioned place preference apparatus, consisting of two chambers (one dark, one light) connected by a hallway, for 15 minutes. The time spent in the light chamber was recorded. During the conditioning phase, mice were first administered saline (i.p.) and kept in the dark chamber for 20 minutes. Three hours later, the analgesic retigabine was administered (10 mg/kg; i.p.) and after 10 mins, the mice were placed in the light chamber for 20 minutes. The conditioning was completed over four consecutive days. On the fifth day, the mice were again allowed to freely explore the apparatus for 15 minutes without any retigabine/saline injections. Data are presented as the difference in time spent in the light (retigabine-paired) chamber during the drug-free test on day five minus time spent in the light chamber at baseline (pre-conditioning phase).

Western Blotting

Within 4 h of the final dose, mice were deeply anesthetized with Beuthanasia-D and then transcardially perfused with ice-cold saline. In some experiments, blood was collected via cardiac puncture prior to perfusion. The ipsilateral and contralateral L4/5 dorsal root ganglia (DRG) were isolated and rapidly frozen for subsequent analysis. DRG from 3 mice were pooled within groups (treatment, lateralization, sex) to ensure that sufficient protein could be obtained for analysis. Nuclear fractions were isolated with a NE-PER Nuclear and Cytoplasmic Extraction Kit, according to manufacturer instructions. Western blotting was performed as previously described. Nuclear proteins were subjected to NuPAGE Bis-Tris (4 to 12%) gel electrophoresis under reducing conditions. After transfer to nitrocellulose membranes, non-specific binding sites were blocked with Superblock buffer for 1 hour at room temperature. Membranes were incubated overnight at 4° C. with primary anti-NRF2 antibody (1:1000; rabbit polyclonal IgG) and anti-histone H3 antibody (1:2000; rabbit polyclonal IgG) (loading control). The membranes were then washed with phosphate buffered saline containing 0.10% Tween-20 and probed with horseradish peroxidase secondary antibody (1:5,000; goat polyclonal IgG) in blocking buffer containing 0.1% Tween-20 for 1 h at room temperature. After washing with 1×PBS containing 0.1% Tween-20, membranes were developed with enhanced chemiluminescent substrate. Images were acquired using ImageQuant LAS 4000. Densitometry analysis was performed using ImageQuant TL software. Data were normalized to loading control (histone H3).

Leukocyte Counting

Leukocytes from cardiac blood were stained with Turk's solution according to manufacturer instructions, and manually counted on a hemocytometer by an experimenter who was blinded to treatment conditions.

Statistics

Differences between in vitro concentration-response relationships were determined by comparing the slopes of fitted functions for shared parameters. Linear models were selected as the pharmacologically-relevant concentrations tested were within the linear range of the concentration-response functions. Mechanical allodynia was analyzed as the interpolated 50% thresholds (absolute threshold). One-way ANOVA was used to confirm that there were no baseline differences in absolute thresholds or dynamic scores between treatment groups. Differences between treatment groups for behavior were determined using repeated measures two-way ANOVA, followed by Tukey's or Sidaks post hoc tests. Biochemical data were analyzed by one- or two-way ANOVA, followed by Tukey's post hoc tests. Results are expressed as mean±standard deviation (SD). P<0.05 was considered statistically significant.

Results

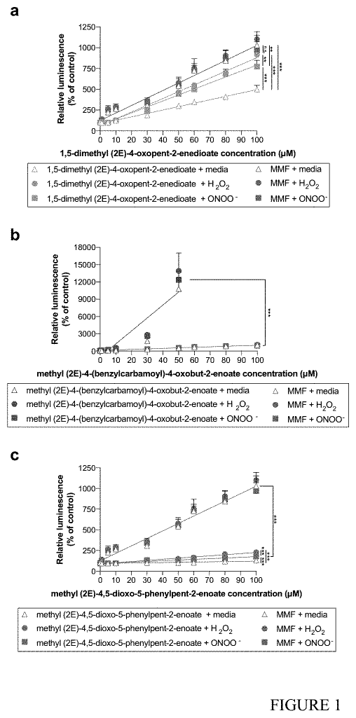

Results from the in vitro assay are presented in FIG. 1. MMF increased luciferase activity in a concentration dependent manner (P<0.001), and activity was not influenced by H₂O₂ or ONOO⁻ (P=0.256) (FIG. 1). 1,5-dimethyl (2E)-4-oxopent-2-enedioate (Example A) increased NRF2 activity in the presence of H₂O₂ or ONOO⁻, compared to media control (P<0.001) (FIG. 1a). Furthermore, cleaved 1,5-dimethyl (2E)-4-oxopent-2-enedioate had similar activity to MMF (FIG. 1a). Methyl (2E)-4-(benzylcarbamoyl)-4-oxobut-2-enoate (Example C) activated NRF2 to a greater extent than MMF, but did not selectively increase NRF2 activity when co-incubated with H₂O₂ or ONOO⁻, compared to media (P=0.288) (FIG. 1b). Methyl (2E)-4,5-dioxo-5-phenylpent-2-enoate (Example D) increased NRF2 activity in the presence of H₂O₂ or ONOO⁻, compared to media control, but at lower levels than MMF (P<0.001).

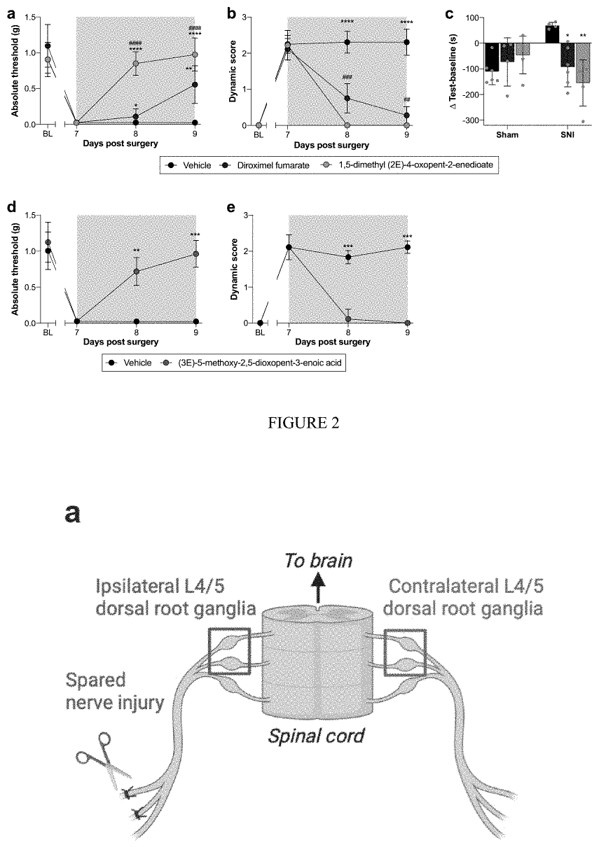

1,5-dimethyl (2E)-4-oxopent-2-enedioate (Example A) was selected for initial testing in vivo as it was responsive to both hydrogen peroxide and peroxynitrite, had no toxicity in the concentration range tested, and had greater efficacy than methyl (2E)-4,5-dioxo-5-phenylpent-2-enoate (Example D). In the spared nerve injury (SNI) model of neuropathic pain, 1,5-dimethyl (2E)-4-oxopent-2-enedioate reversed tactile (FIG. 2a, time×treatment: F_{4, 66}=71.05, P<0.0001; time: F_{1.48, 48.98}=162.70, P<0.0001; treatment: F_{2, 33=124.50}, P<0.0001) and dynamic allodynia (FIG. 2b, time×treatment: F_{4, 66}=107.50, P<0.0001; time: F_{1.70, 56.08}=356.60, P<0.0001; treatment: F_{2, 33}=164.80, P<0.0001), while vehicle did not alter paw withdrawal responses. On both measures, 1,5-dimethyl (2E)-4-oxopent-2-enedioate had greater efficacy than diroximel fumarate (P<0.01). Neither compound altered paw responses in the contralateral limb. The results in FIG. 2c shows that nerve-injured mice increased the time spent in the light chamber paired with retigabine, indicating ongoing pain. Sham-operated mice maintain a preference for the dark chamber. Nerve-injured mice that were treated with 1,5-dimethyl (2E)-4-oxopent-2-enedioate or diroximel fumarate did not develop a preference for the retigabine-paired light chamber, indicating that these mice no longer experienced spontaneous pain. (3E)-5-methoxy-2,5-dioxopent-3-enoic acid (Example J) also reversed tactile (FIG. 2d, time×treatment: F_{3, 30}=32.03, P<0.0001; time: F_{1.82, 18.20}=117.5, P<0.0001; treatment: F_{1, 10}=44.35, P<0.0001) and dynamic allodynia (FIG. 2e, time×treatment: F_{3, 30}=82.36, P<0.0001; time: F_{1.70, 17.00}=196.3, P<0.0001; treatment: F_{1, 10}=218.4, P<0.0001), while vehicle did not alter paw withdrawal responses.

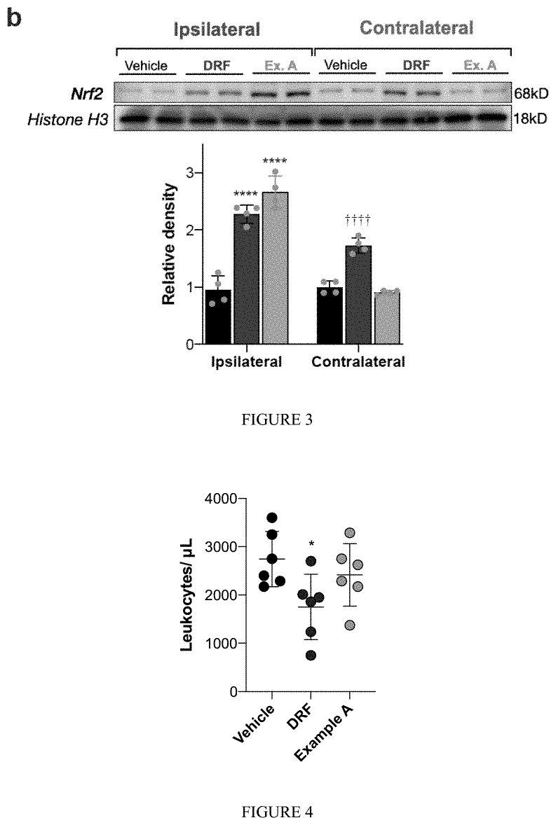

Unilateral peripheral nerve injury induces nitro-oxidative stress in the ipsilateral dorsal root ganglia. We therefore tested whether 1,5-dimethyl (2E)-4-oxopent-2-enedioate (Example A) selectively activated the NRF2 pathway in the ipsilateral DRG as evidence of site-specific cleavage (FIG. 3a). NRF2 nuclear translocation, measured by Western blot, occurred only in the ipsilateral DRG (FIG. 3b). In contrast, diroximel fumarate non-selectively induced NRF2 nuclear translocation in both the ipsilateral and contralateral DRG (FIG. 3b).

Aligning with the targeted activation of the NRF2 pathway, 1,5-dimethyl (2E)-4-oxopent-2-enedioate (Example A) did not cause a decline in absolute leukocyte count, FIG. 4.

1. A method of treating disorders associated with oxidative stress including the step of administering to a subject in need thereof a compound of formula (I): 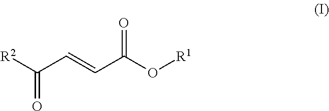
wherein
R¹ is selected from C₁-C₃ alkyl;
R² represents: 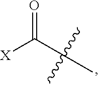
where X is selected from OH, OX¹, CH₂C(O)X², C(O)X², CHCHC(O)X², optionally substituted aryl, optionally substituted heteroaryl, optionally substituted heterocyclyl, optionally substituted alkyl, optionally substituted alkoxy, optionally substituted amino and optionally substituted thio; and
wherein X¹ is selected from optionally substituted aryl, optionally substituted heteroaryl, optionally substituted heterocyclyl, optionally substituted alkyl, optionally substituted amino and optionally substituted thio; and
wherein X² is selected from OH, optionally substituted aryl, optionally substituted heteroaryl, optionally substituted heterocyclyl, optionally substituted alkyl, optionally substituted alkoxy, optionally substituted amino and optionally substituted thio;
or a pharmaceutically acceptable salt, solvate, or isomer thereof.

2. A method according to claim 1 wherein the treatment is selected from treating vascular diseases, high cholesterol, stroke, heart failure, and hypertension; cancer; neurodegenerative diseases; diabetes; pain disorders; multiple sclerosis; kidney disease; rheumatoid arthritis; sepsis; respiratory distress syndrome; and metabolic disorders, such as mitochondrial diseases, lipid metabolism disorders, and DNA repair-deficiency disorders, COPD, and CKD.

3. A method according to claim 1, wherein treatment of the disorder associated with oxidative stress is through site directed target engagement of the NRF2 pathway.

4. A method according to claim 1, wherein the treatment is the treatment of pain, preferably neuropathic pain.

5. A method according to claim 4, wherein the treatment of neuropathic pain is the treatment of peripheral neuropathic pain.

6. A method according to claim 1, wherein the compound of formula (I) is as represented by formula (Ia): 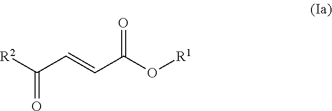
where R¹ is C₁-C₃ alkyl and
where R² is selected from:
a) 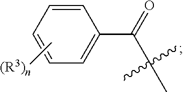
b) optionally substituted C₁-C₁₂ alkyl 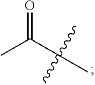 [SMILES: *C(C)=O]
c) optionally substituted heterocyclyl 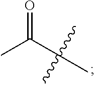 [SMILES: *C(C)=O]
d) optionally substituted heteroaryl 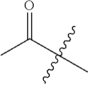 [SMILES: *C(C)=O]
e) 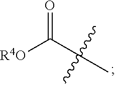
f) 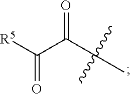
g) 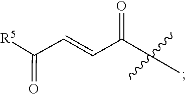
h) 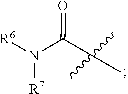
and
i) 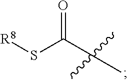
wherein R³ is selected from OH, CI, F, CF₃, CN, OCF₃, optionally substituted alkyl, optionally substituted alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, optionally substituted cycloalkyl, optionally substituted cycloalkyloxy, optionally substituted sulfinyl, and optionally substituted sulfonyl.
n is an integer selected from 0 to 3;
R⁴ is selected from H, optionally substituted alkyl, optionally substituted aryl, optionally substituted heteroaryl, and optionally substituted heterocyclyl;
R⁵ is selected from optionally substituted alkyl, optionally substituted alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, and optionally substituted cycloalkyl;
R⁶ and R⁷ are independently selected from H, optionally substituted C₁-C₆ alkyl, optionally substituted aryl and optionally substituted arylalkyl; and
R⁸ is selected from optionally substituted alkyl, optionally substituted aryl, and optionally and substituted arylalkyl.

7. A method according to claim 6, wherein the compound of Formula (Ia) is selected from a compound of Formula (Ia)(a-e).

8. A method according to claim 6, wherein the compound of Formula (Ia) is a compound of Formula (Ia)(a).

9. A method according to claim 6, wherein the compound of Formula (Ia) is a compound of Formula (Ia)(b).

10. A method according to claim 6, wherein the compound of Formula (Ia) is a compound of Formula (Ia)(c).

11. A method according to claim 6, wherein the compound of Formula (Ia) is a compound of Formula (Ia)(d).

12. A method according to claim 6, wherein the compound of Formula (Ia) is a compound of Formula (Ia)(e).

13. A method according to claim 6, wherein the compound of Formula (Ia) is a compound of Formula (Ia)(f).

14. A method according to claim 6, wherein the compound of Formula (Ia) is a compound of Formula (Ia)(g).

15. A method according to claim 6, wherein the compound of Formula (Ia) is a compound of Formula (Ia)(h).

16. A method according to claim 6, wherein the compound of Formula (Ia) is a compound of Formula (Ia)(i).

17. A method according to claim 6, wherein the compound of formula (Ia) is compound of (Ia)(e): 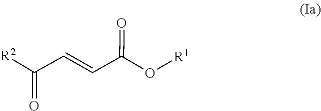
or pharmaceutically acceptable salts thereof:
where R¹ is C₁-C₃ alkyl and
where R² is:
e) 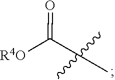
wherein R⁴ is selected from H, optionally substituted alkyl, optionally substituted aryl, optionally substituted heteroaryl, and optionally substituted heterocyclyl.

18. A method according to claim 17, wherein R⁴ is selected from H, C₁-C₈ substituted alkyl, optionally substituted aryl, optionally substituted heteroaryl, and optionally substituted heterocyclyl.

19. A method according to claim 18, wherein R⁴ is selected from H and C1-C8 alkyl or wherein R⁴ is selected from H and C1-C3 alkyl or wherein R⁴ is H or C1-C2 alkyl or R⁴ is C1-C2 alkyl, or R⁴ is C1 alkyl, or R⁴ is C2 alkyl or R⁴ is H.

20. A method according to claim 19 wherein R⁴ is a salt form selected from K, Li, Na.

21. A method according to claim 1 wherein the compound of Formula (I) is selected from:

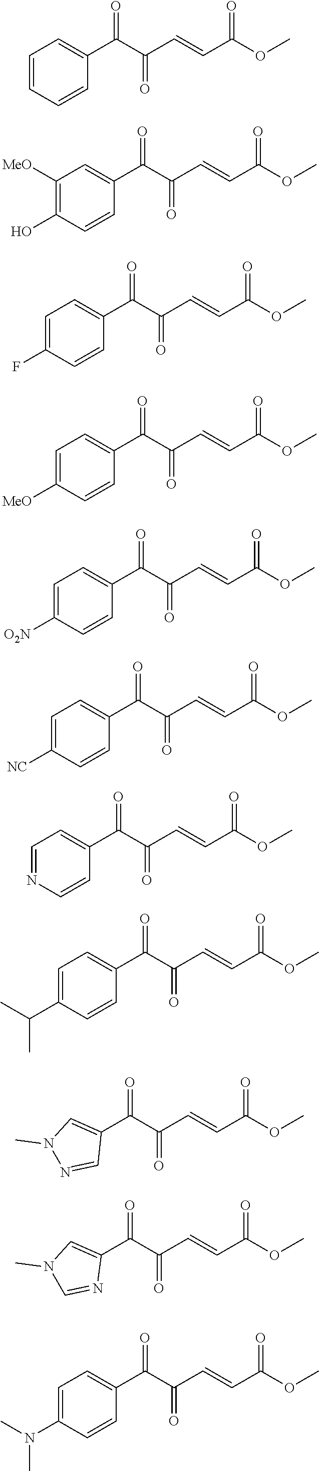

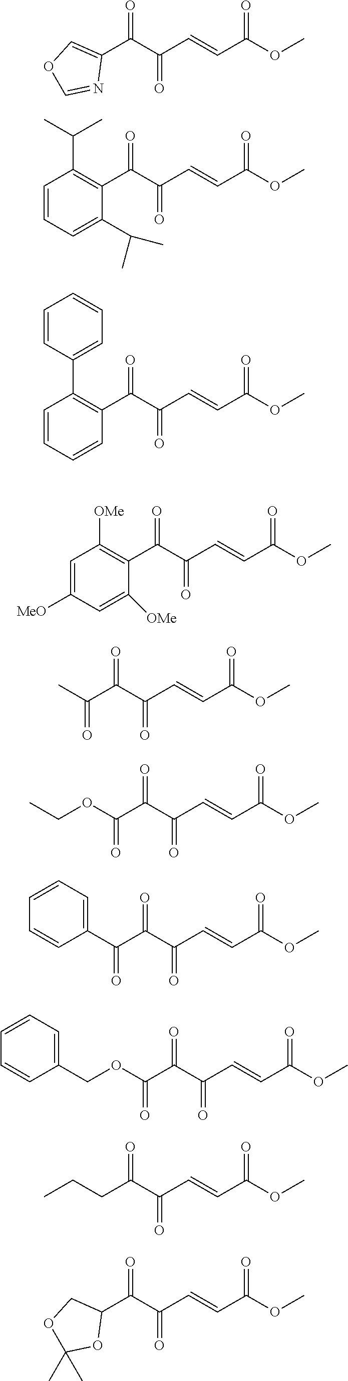

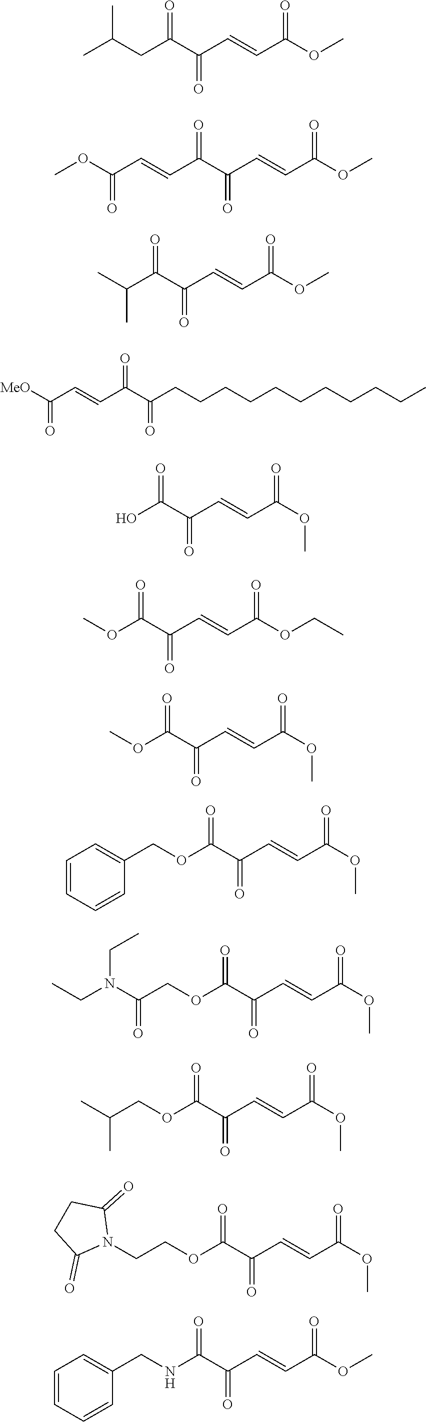

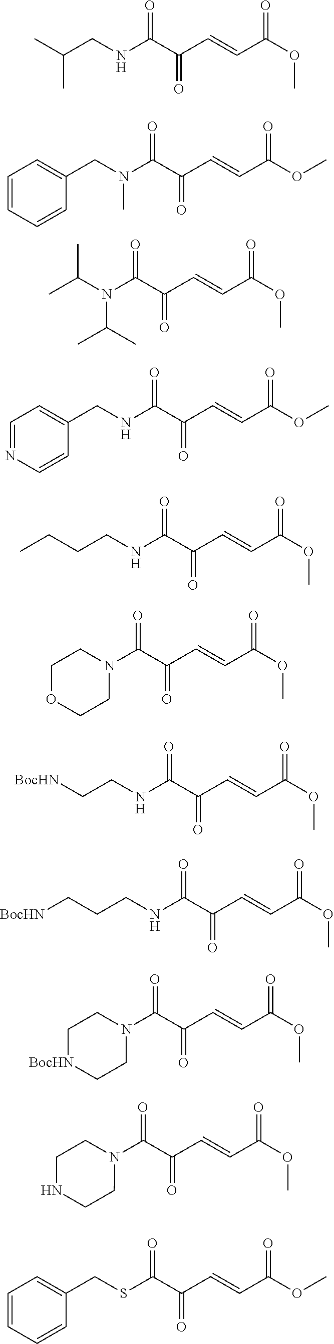

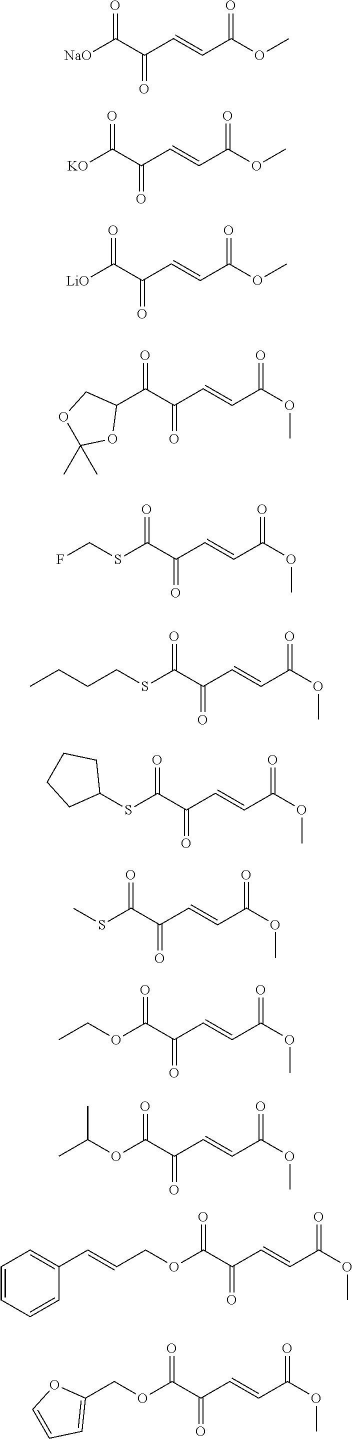

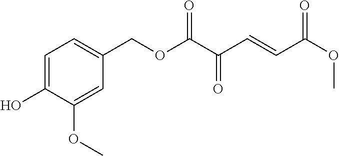 [SMILES: COC(=O)/C=C/C(=O)C(=O)OCc1ccc(O)c(OC)c1]

22. A compound of formula (II) 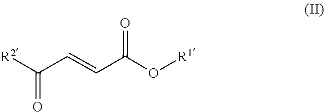
or a pharmaceutically acceptable salt thereof:
where R^{1′} is C₁-C₃ alkyl and
where R^{2′} is selected from:
a′) 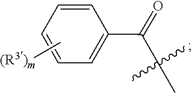
b′) optionally substituted C₂-C₁₀ alkyl 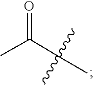 [SMILES: *C(C)=O]
c′) optionally substituted heterocyclyl 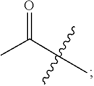 [SMILES: *C(C)=O]
d′) optionally substituted heteroaryl 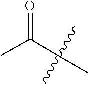 [SMILES: *C(C)=O]
e′) 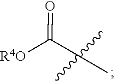
f′) 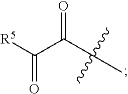
g′) 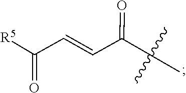
h′) 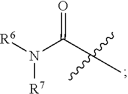
and
i′) 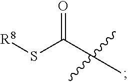
wherein R^{3′} is selected from OH, CI, F, CF₃, CN, OCF₃, optionally substituted alkyl, optionally substituted alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, optionally substituted cycloalkyl, optionally substituted cycloalkyloxy, optionally substituted sulfinyl, and optionally substituted sulfonyl;
m is an integer selected from 1 to 3;
provided that when m is 1, R^{3′} is not methyl, OH, NO₂ or methoxy;
R^{4′} is selected from optionally substituted C₄-C₈ alkyl, optionally substituted aryl, optionally substituted heteroaryl, and optionally substituted heterocyclyl;
R^{5′} is selected from optionally substituted alkyl, optionally substituted C₂-C₆ alkoxy, optionally substituted heteroaryl, optionally substituted aryl, optionally substituted acyl, optionally substituted heterocyclyl, optionally substituted alkenyl, optionally substituted heteroaryloxy, optionally substituted aryloxy, optionally substituted heterocyclyloxy, optionally substituted alkenyloxy, and optionally substituted cycloalkyl;
R^{6′} and R^{7′} are independently selected from H, optionally substituted C₁-C₆ alkyl, optionally substituted aryl and optionally substituted arylalkyl; provided that when one of R^{6′} and R^{7′} is H the other is not benzyl; and
R^{8′} is selected from optionally substituted alkyl, optionally substituted aryl, and optionally and substituted arylalkyl.

23. A compound according to claim 22 wherein the compound is selected from compounds of Formula (II)(e′) wherein R4′ is selected from optionally substituted C4-C8 alkyl, optionally substituted aryl, optionally substituted heteroaryl, and optionally substituted heterocyclyl.

24. A compound according to claim 22 wherein the compound is selected from compounds of Formula (II)(e′) wherein R4′ is selected from optionally substituted aryl.

25. A compound according to claim 22 wherein the compound is selected from compounds of Formula (II)(e′) wherein R4′ is selected from optionally substituted heteroaryl.

26. A compound according to claim 23 wherein the compound is selected from compounds of Formula (II)(e′) wherein R4′ is selected from optionally substituted heterocycyl.

27. A compound according to claim 22 selected from: 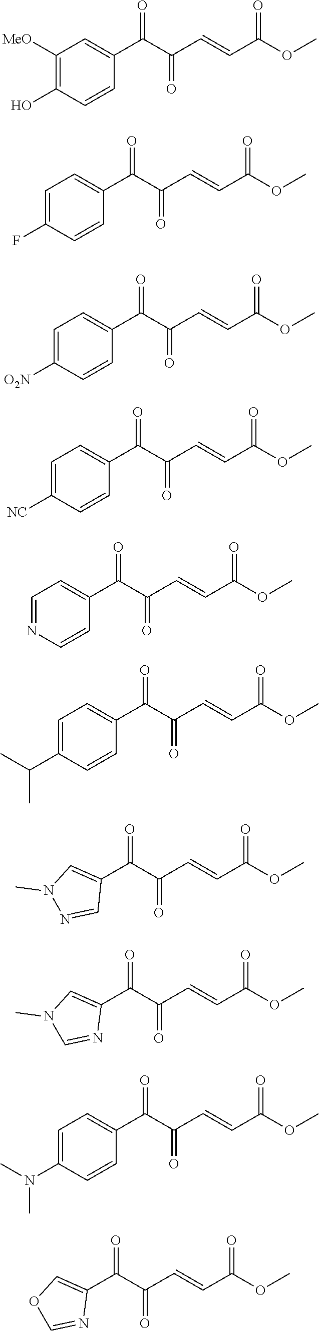 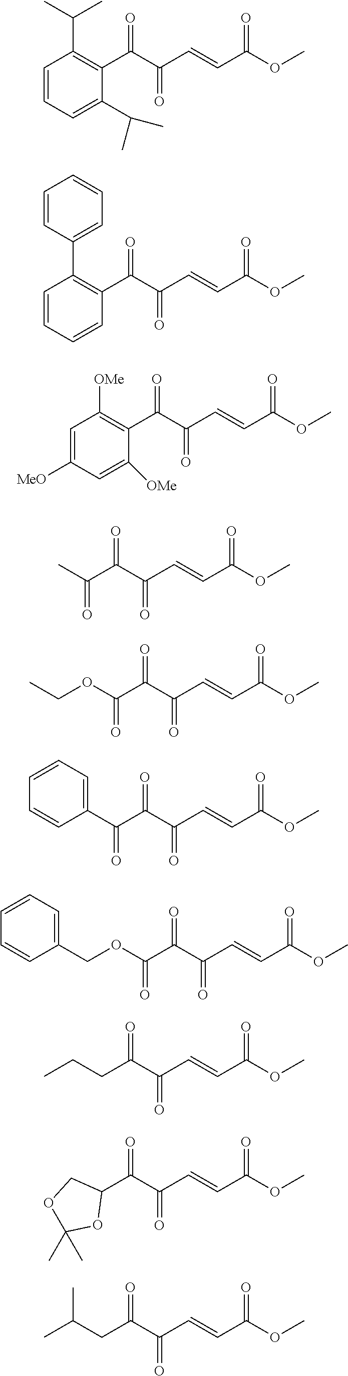 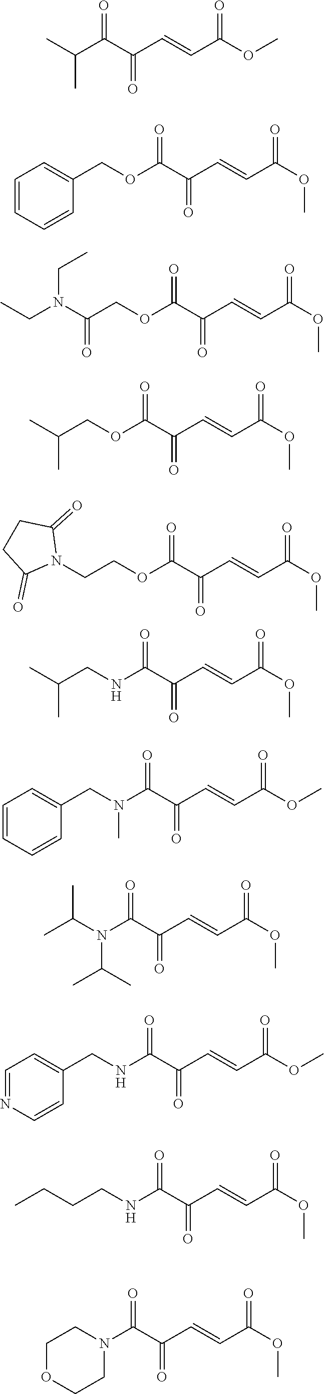 [chemical structure]
